(19) United States
(12) Patent Application Publication
(10) Pub. No.: US 2007/0005127 A1
(43) Pub. Date: Jan. 4, 2007

(54) Hinged tissue implant and related methods and devices for delivering such an implant

(76) Inventors: Peter Boekstegers, Diessen (DE); Patrick T. Cahalan, Cape Coral, FL (US); Laurence A. Roth, Windham, NH (US); Jianlu Ma, Maple Grove, MN (US); Earl F. Bardsley, Newton, MN (US); Justin A. Callaway, Bedford, NH (US); James F. Duronio, Westford, MA (US); Steve W. Opolski, Carlisle, MA (US); Wendy R. Graczyk, Allston, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/154,710

(22) Filed: Jun. 17, 2005

(51) Int. Cl. A61F 2/06

(52) U.S. Cl. 623/1.16; 623/1.11

(57) ABSTRACT

A device for treating a heart may comprise a myocardial section configured to be positioned in a heart wall between a coronary vessel and a chamber of the heart and a vessel section configured to be positioned in the coronary vessel. The vessel section may be connected to the myocardial section and be configured to articulate relative to the myocardial section. The myocardial section and the vessel section may form an integral, single piece structure.

### FIELD OF THE INVENTION

Exemplary embodiments of the present invention relate generally to a medical implant for inserting into body tissue and a method of delivering and using such a medical implant. Exemplary embodiments of the present invention also may relate to an implant configured to provide flow communication between blood-containing coronary structures, such as, for example, between a heart chamber and a coronary vessel or between two coronary vessels.

### BACKGROUND

An implant for insertion into body tissue may have various uses, such as providing flow communication between two body parts, delivering drugs into a body part, or serving as a sensor, controller, or monitoring device, for example. Without limiting the scope of the present invention, the following paragraphs describe an exemplary use of an implant, such as a stent or conduit, for example, to treat blockages in coronary vessels. The examples discussed below do not constitute a limitation on the scope and applications of the present invention.

Coronary artery disease may be treated with several approaches. Coronary arteries, as well as other coronary vessels, frequently become clogged with plaque which, at the very least, can reduce blood and oxygen flow to the heart muscle (myocardium). The plaque also may impair the efficiency of a heart's pumping action and lead to heart attack or death. In some cases, these coronary arteries can be unblocked through noninvasive techniques, such as, for example, performing balloon angioplasty or stenting a vessel to provide a blood passageway. In more difficult cases, performing a surgical bypass of the blocked vessel may be necessary.

One conventional treatment for a clogged coronary artery is a coronary bypass operation wherein one or more venous segments are inserted between the aorta and the coronary artery. The inserted venous segments or transplants bypass the clogged portion of the coronary artery and thus provide a free and unobstructed flow communication of blood between the coronary artery and the heart. Such conventional coronary artery bypass surgery, however, may be expensive, time-consuming, and traumatic to a patient. Hospital stay subsequent to surgery and convalescence generally is prolonged. Furthermore, many patients may not be suitable surgical candidates due to other concomitant illnesses.

An alternative to coronary artery bypass, angioplasty, and vessel stenting includes providing a flow passage in the myocardial wall between the left ventricle and the coronary artery. The passage may be provided at a point downstream of the blockage. In this technique, a portion of the blood from the left ventricle flows directly through the passage in the myocardial wall and into the artery downstream of the blockage. A variation of this technique includes placing a stent in the heart wall to provide the blood flow passage between the left ventricle and coronary artery.

FIG. 1 illustrates a partial cross-sectional view of a heart 10 having an implant in the form of a stent 12 disposed in a heart wall MYO. As shown, the stent 12 extends between a left ventricle LV and a coronary artery CA. The coronary artery CA has a posterior wall 14 and an anterior wall 16. The stent 12 is positioned at a point in the coronary artery CA downstream of an occlusion or blockage BL of the coronary artery CA. In general, the occlusion or blockage BL in the coronary artery CA can be partial or full so as to inhibit or completely block the blood flow through the coronary artery CA. When used herein, the terms “occlusion” and “blockage” are intended to include full and partial occlusions or blockages. For the stent 12 positioned between the left ventricle LV and coronary artery CA, connecting positions other than the position depicted in FIG. 1 can be utilized. For example, the stent 12 may form a perpendicular or angled position with respect to the posterior wall 14 of the coronary artery CA or the side of the left ventricle LV. The connection position may be selected so as to avoid interference with various structures in the heart, including the papillary muscles, chordae, and mitral valve, for example.

A variation of this technique includes forming a passage in the myocardial wall between the left ventricle and a coronary vein proximate an occluded coronary artery. The vein may be used to supply oxygenated blood to the heart. A conduit, for example a stent, may be inserted in the passage, and the vein may include a blocking device proximate the passage to restrict blood flow toward the coronary sinus.

As yet a further alternative, the passage may be formed in the myocardial wall between two coronary vessels, such as between a coronary vein and a coronary artery, for example.

A problem that may be encountered when using a stent or other type of implant is migration. Migration of the stent after its insertion may lead to the protrusion of the stent beyond the heart wall, for example, either into the left ventricle or into the blood flow lumen of the coronary vessel, either the coronary artery or coronary vein, for example. Migration may create a risk that the stent may not be positioned so as to establish an unobstructed passageway for blood flow. For example, the stent may extend too far into the lumen of the coronary vessel, blocking blood flow therethrough, or may be displaced from the heart chamber or coronary vessel, hindering blood flow through the myocardial passageway. Migration may also allow portions of the myocardial tissue that surround a passageway to advance toward the passageway and cause the passageway to contract, especially when the myocardial tissue is not sufficiently supported by the stent. The contraction of the passageway may reduce or entirely block blood flow through the stent, thereby rendering the stent less effective in providing an unblocked channel of blood flow to the coronary vessel. In addition to the reduction or complete blockage of blood flow, migration of the stent from a designated location potentially will interfere with other structures in the heart and may pose serious risk of embolization.

Another problem associated with these techniques involves delivery of a myocardial implant. In particular, when using a percutaneous technique, implantation and positioning may prove difficult. For instance, during percutaneous delivery, positioning using X-ray images, fluoroscopy, or the like may not be sufficiently accurate, which may result in the implant being placed too high or too low with respect to the vessel. Moreover, delivery techniques and tools can be relatively complex. In addition, when delivering implants with self-deploying seating mechanisms, such seating mechanisms may be difficult to orient and angle, and there also may be limited feedback regarding positioning of the implant to the physician, for example when using fluoroscopy.

### SUMMARY OF THE INVENTION

Some advantages and purposes of the invention will be set forth in part in the description which follows, and may be obvious from the description, or may be learned by practice of the invention. It should be understood that skilled artisans may practice the invention without having one or more features of any of the objects, aspects, or embodiments described herein. In addition, such features are exemplary and at least some of them are set forth in the detailed description which follows.

Exemplary embodiments of the implant and delivery tool designs may permit percutaneous delivery and proper positioning without great complexity. For example, in exemplary embodiments, the implant may have a relatively small profile so as to facilitate delivery and implantation. Moreover, in exemplary embodiments, the design of delivery tools, such as delivery catheters, for example, to deliver the implant may be simplified.

An exemplary aspect of the invention includes a device for treating a heart. The device may comprise a stent having at least a portion thereof that does not extend completely around the circumference of the stent so as to form a first stent section and a second stent section that are articulatable relative to each other. In an exemplary embodiment, the first stent section may form a myocardial section configured to be positioned in a heart wall between a heart chamber and a coronary vessel. The second stent section may form a vessel section configured to be positioned in a coronary vessel.

Another exemplary aspect of the invention includes a method of treating a heart. The method may include providing a stent comprising a plurality of stent cells, wherein at least part of a stent cell does not extend completely around the circumference of the stent so as to permit a first stent section and a second stent section to articulate relative to each other The method may further include delivering the stent to a location proximate to a heart wall, inserting the first section in a heart wall between a coronary vessel and a chamber of the heart, and inserting the second section in the coronary vessel.

In another exemplary aspect, the invention includes a delivery system. The delivery system may comprise a catheter having a proximal end portion and a distal end portion. The distal end portion may comprise a first lumen and a second lumen substantially adjacent the first lumen. The first lumen and the second lumen may form a branched configuration so as to permit independent movement of the first lumen and the second lumen.

Yet a further exemplary embodiment of the invention includes a method of making an implant. The method may include providing a stent comprising a plurality of stent cells and forming at least one stent cell such that a circumferential portion of at least part of the stent cell is missing so as to form a first stent section and a second stent section that are configured to articulate relative to each other.

Yet another exemplary aspect of the invention includes a device for treating a heart comprising a stent comprising a first stent section, a second stent section, and a third stent section connecting the first and second stent sections. Each of the first stent section and the second stent section may include at least one stent cell, and the connecting section may include a strut of the stent (e.g., a segment of the stent) extending around only a portion of a circumference of the stent so that the first and second stent sections articulate relative to each other. In an exemplary aspect, a stent cell may include one of a plurality of repeating annular segments of the stent. In a further exemplary aspect, the connecting section may have a length less than a length of a stent cell.

According to another exemplary aspect, the invention may include a device for treating a heart comprising a stent comprising a plurality of stent cells, wherein at least one stent cell is missing at least a circumferential portion thereof so as to permit a first stent section and a second stent section to articulate relative to each other.

According to an exemplary aspect, the implant may be in the form of a stent. The myocardial section of the implant may be inserted in the heart wall so as to place the heart chamber and coronary vessel in flow communication with each other. The myocardial section may be inserted in a heart wall between a left ventricle and either a coronary artery or a coronary vein, for example.

Another exemplary embodiment of the invention includes a device for treating a heart. The device includes a stent having a portion thereof that does not extend completely around the circumference of the stent so as to form a first stent section and a second stent section that articulate relative to each other.

In various embodiments, the invention may include one or more of the following aspects: the first stent section may include a myocardial section configured to be positioned in a heart wall between a coronary vessel and a chamber of the heart and the second stent section may include a vessel section configured to be positioned in the coronary vessel; the myocardial section may be configured to provide flow communication between the heart chamber and the coronary vessel when the stent is positioned in the heart wall; the myocardial section may be configured to be positioned in the heart wall between a left ventricle and one of a coronary artery and a coronary vein; a covering over at least a portion of the stent; the covering may include one of a polymer, a metal, and a tissue; the first section and the second section may be connected to each other via a hinged section; the portion of the stent that does not extend completely around the circumference of the stent may form the hinged section; the hinged section may have a length equal to approximately half of a length of a stent cell; the portion may extend from approximately 10 degrees to approximately 90 degrees around the circumference of the stent; the stent may be expandable; the vessel section may be configured to achieve a diameter approximately equal to a diameter of the vessel lumen when the vessel section is positioned in the coronary vessel; the device may be configured to be percutaneously delivered and implanted in the heart; and the device may be configured to be surgically delivered and implanted in the heart.

A further exemplary embodiment of the invention includes a method of treating a heart. The method includes providing a stent having a portion that does not extend completely around the circumference of the stent so as to form a first stent section and a second stent section that articulate relative to each other, delivering the stent to a location proximate to a heart wall, inserting the first stent section in a heart wall between a coronary vessel and a chamber of the heart, and inserting the second stent section in the coronary vessel.

In various embodiments, the invention may include one or more of the following aspects: forming a passageway in the heart wall between the coronary vessel and the heart chamber and inserting the first section into the passageway; flowing blood through the stent between the heart chamber and the coronary vessel after inserting the stent; delivering the stent may include delivering the stent to the location proximate to the heart wall via a catheter; inserting the first stent section may include inserting the first stent section in the heart wall between a left ventricle and one of a coronary artery and a coronary vein; expanding the stent; expanding the stent may include expanding the stent via a balloon; expanding the stent may include allowing the stent to self-expand; delivering the stent may include percutaneously delivering the stent; and delivering the stent may include delivering the stent through a lumen of the coronary vessel.

Still another exemplary embodiment of the invention includes a delivery system. The delivery system includes a catheter having a proximal end portion and a distal end portion. The distal end portion includes a first shaft defining a first lumen and a second shaft defining a second lumen, the second shaft being substantially adjacent the first shaft. The first shaft and the second shaft form a branched configuration so as to permit independent movement of at least portions of the first shaft and the second shaft.

In various embodiments, the invention may include one or more of the following aspects: a first balloon carried by the first shaft and a second balloon carried by the second shaft; the proximal end portion may include the first shaft and the second shaft connected to each other; the proximal end portion may include a third shaft defining a third lumen; the third lumen may be in flow communication with a first balloon carried by the first shaft and a second balloon carried by the second shaft; and a first guide wire configured to exit the first shaft and a second guidewire configured to exit the second shaft.

A still further exemplary embodiment of the invention includes a method of delivering an implant to a heart wall between a heart chamber and a coronary vessel. The method includes advancing a first shaft of a catheter into the heart wall, the first shaft carrying a first section of the implant, advancing a second shaft of the catheter into the coronary vessel, the second shaft carrying a second section of the implant, and positioning the implant such that the first section of the implant is placed in the heart wall and the second section of the implant is placed in the lumen of the coronary vessel.

In various embodiments, the invention may include one or more of the following aspects: inserting a second guidewire through a lumen of the coronary vessel and inserting a first guidewire through the lumen of the coronary vessel and into the heart wall; advancing the first shaft includes advancing the first shaft over the first guidewire and advancing the second shaft includes advancing the second shaft over the second guidewire; positioning the implant includes positioning the implant such that the implant places the heart chamber in flow communication with the lumen of the coronary vessel; expanding the first and second sections of the implant; expanding the first and second sections of the implant includes expanding the first section via a first balloon carried by the first shaft and expanding the second section via a second balloon carried by the second shaft; the first shaft may also carry the second section of the implant; carrying the implant on the catheter such that the first and second sections are folded relative to each other; the implant may be a stent; the heart chamber may be a left ventricle; the coronary vessel may be chosen from a coronary vein and a coronary artery; bending the first and second sections relative to each other during the positioning of the implant; the positioning may include positioning the second section such that a longitudinal axis of the second section is substantially parallel to a longitudinal axis of the lumen of the coronary vessel; at least one of the first shaft and the second shaft may be carrying a third section of the implant connected to one of the first section and the second section; positioning the implant such that the third section of the implant is placed in the lumen of the coronary vessel; the third section may not be connected to the first section; the third section may not be connected to the second section; carrying the implant on the catheter such that at least two of the first, second, and third sections are folded relative to each other; bending at least two of the first, second, and third sections relative to each other during the positioning of the implant; the positioning may include positioning the third section such that a longitudinal axis of the third section is substantially parallel to a longitudinal axis of the lumen of the coronary vessel.

Yet another exemplary embodiment of the invention includes a device for treating a heart. The device includes a stent comprising a first stent section, a second stent section, and a third stent section connecting the first and second stent sections. Each of the first stent section and the second stent section includes at least one stent cell. The connecting section includes at least one stent segment extending around only a portion of a circumference of the stent so that the first and second stent sections articulate relative to each other.

In various embodiments, the invention may include one or more of the following aspects: a stent cell may include one of a plurality of repeating annular segments of the stent; and the connecting section may have a length less than a length of a stent cell.

A yet further exemplary embodiment of the invention includes a device for treating a heart. The device includes a stent structure having a circumferential portion missing therefrom so as to form a first section of the stent structure and a second section of the stent structure that are configured to articulate relative to each other.

Another exemplary embodiment of the invention includes a device for treating a heart. The device includes a first stent section, a second stent section connected to the first stent section and configured to articulate relative to the first stent section, and a third stent section connected to the first stent section, unconnected to the second stent section, and configured to articulate relative to the first stent section. The first, second, and third stent sections may be configured to form a substantially T-shaped structure.

In various embodiments, the invention may include one or more of the following aspects: the first stent may be connected to the second stent section via a connector; the first and second stent sections may be configured to articulate about the connector; the third stent section may be connected to the first stent section via a connector; the first and third stent sections may be configured to articulate about the connector; the first stent section may be configured to be positioned in one of a coronary vessel and a heart wall between a coronary vessel and a heart chamber; the first stent section may be configured to be positioned in a heart wall between a coronary vessel and a heart chamber; the second and third stent sections may be configured to be positioned in the coronary vessel; the first and second stent sections may be configured to be positioned in a coronary vessel; and the third section may be configured to be positioned in a heart wall between the coronary vessel and a heart chamber.

A further exemplary embodiment of the invention includes a device for treating a heart. The device includes a first stent section having a lumen. The first stent section includes an open first end, an open second end, and a hole between the first and second ends. The device also includes a second stent section extending through the hole. The second stent section includes a first portion extending within the lumen of the first stent section and a second portion extending from the hole outside of the lumen.

In various embodiments, the first and second stent sections may form a substantially T-shaped structure.

Still another exemplary embodiment of the invention includes a device for treating a heart. The device includes a first stent section and a second stent section connected to, and configured to articulate relative to, each other, and a third stent section and a fourth stent section connected to, and configured to articulate relative to, each other. One of the third stent section and the fourth stent section is configured to be disposed within a lumen of one of the first stent section and the second stent section.

In various embodiments, a longitudinal axis of the one of the third stent section and the fourth stent section may be configured to be substantially parallel to a longitudinal axis of the one of the first stent section and the second stent section.

A still further exemplary embodiment of the invention includes a device for treating a heart. The device includes a first stent section and a second stent section connected to the first stent section via a connector and configured to articulate relative to the first stent section about the connector.

In various embodiments, the invention may include one or more of the following aspects: the connector may be at least one of a ring, a loop, a wire, a strip, a string, a cable, a suture, and a rope; each of the first stent section and the second stent section may include a U-shaped section; the connector may be disposed about the U-shaped section of each of the first stent section and the second stent section; each of the first stent section and the second stent section may include a hole; the connector may be disposed through the hole of each of the first stent section and the second stent section; the connector may be a notched region; the connector may be attached to at least one of the first stent section and the second stent section; and the connector may be at least two connectors.

Yet another exemplary embodiment of the invention includes a method of placing a first guidewire in a coronary vessel and a second guidewire in a heart wall. The method includes providing the first guidewire, the second guidewire, a puncture tool including a lumen and a side hole, and a guide tool including a guide, advancing the first guidewire through a puncture site and into the coronary vessel, advancing the guide tool through the puncture site and into the coronary vessel via the first guidewire, advancing the puncture tool through the guide, the coronary vessel, and the heart wall into a heart chamber, advancing the second guidewire through the heart wall and into the heart chamber via the guide tool, the side hole, and the lumen, and removing the puncture tool.

In various embodiments, the invention may include one or more of the following aspects: the guide tool may include a first section and a second section; each of the first and second sections may include the guide; advancing the guide tool may include advancing the second section through the puncture site and into the coronary vessel via the first guidewire until a portion of the first section is substantially flush with an outer surface of the coronary vessel; the guides on the first and second sections may be substantially aligned; the puncture tool may include a slit in communication with the lumen and the side hole; removing the puncture tool may include removing the puncture tool such that the second guidewire exits the lumen via the slit; the puncture tool may include markers on an outer surface; and determining a thickness of the heart wall via the markers.

A yet further exemplary embodiment of the invention includes a puncture tool configured to assist the advancement of a guidewire into a heart wall. The method includes an elongate body including a distal end configured to puncture tissue, the elongate body defining a lumen, a hole in a side of the elongate body, and a slit extending from the hole to the distal end.

In various embodiments, the invention may include one or more of the following aspects: markers on an outer surface of the elongate body and the markers may be configured to assist in determining a thickness of the heart wall.

### BRIEF DESCRIPTION OF THE DRAWINGS

The accompanying drawings, which are incorporated in and constitute a part of this specification, illustrate exemplary embodiments of the invention. Those embodiments, together with the following description, serve to explain certain principles and provide a further understanding of the invention. In the drawings,

FIG. 1 is a partial cross-sectional view of a heart with a stent disposed in the heart wall between the left ventricle and coronary artery downstream of an occlusion in the coronary artery;

FIG. 2 is a partial cross-sectional view of a heart with an exemplary embodiment of a two-section stent disposed in the heart wall between the left ventricle and a coronary vessel;

FIG. 3 is a partial cross-sectional view of a heart with an exemplary embodiment of a two-section stent disposed in the heart wall between the left ventricle and a coronary vein and a venous blocking device disposed in the coronary vein;

FIG. 4 is a side view of an exemplary embodiment of a stent in an undeployed state;

FIG. 5 is a side view of the stent of FIG. 4 in a deployed state;

FIG. 6 is a partial side view of an exemplary embodiment of a delivery system for delivering a hinged implant;

FIG. 6A is a partial side view of another exemplary embodiment of a delivery system for delivering a hinged implant;

FIG. 7 is a partial side view of the stent of FIGS. 4 and 5 and carried by the delivery system of FIG. 6 according to an aspect of the invention;

FIG. 8 is a step of an exemplary embodiment of delivering a hinged implant according to an aspect of the invention;

FIG. 9 is another step of an exemplary embodiment of delivering a hinged implant using the delivery system of FIGS. 6 and 7;

FIG. 10 is a partial side view of the stent of FIGS. 4 and 5 carried by an exemplary embodiment of a delivery system; and

FIG. 11 is another step of an exemplary embodiment of delivering a hinged implant using the delivery system of FIG. 10;

FIG. 12 is a partial cross-sectional view of a heart with a hinged implant disposed in the heart wall after delivery of the implant using the delivery system and method of FIGS. 6-9;

FIG. 13 is a side schematic view of a stent having three sections, according to another embodiment of the invention;

FIG. 14 is a side schematic view of a stent having three sections, according to a further embodiment of the invention;

FIG. 15 is a side schematic view of a stent including a ring, according to still another embodiment of the invention;

FIGS. 16A and 16B are schematic views of different embodiments including one stent disposed in another stent, according to a still further embodiment of the invention;

FIG. 17 is a schematic view of a portion of one stent disposed in another stent, according to yet another embodiment of the invention;

FIG. 18 is a side schematic view of a stent having three sections, according to a yet further embodiment of the invention;

FIGS. 19A-19G are schematic views of connectors connecting stent sections, according to various embodiments of the invention;

FIGS. 20A-20B show steps of an exemplary embodiment of delivering a stent using guidewires deployed using the method shown in FIGS. 21A-21D;

FIGS. 20C-20D show steps of an exemplary embodiment of delivering a stent using guidewires deployed using the method shown in FIGS. 21A-21D;

FIGS. 21A-21 D are steps in a method of deploying guidewires in a coronary vessel and a heart wall using the guidewire placement devices of FIGS. 22A-22B, according to an exemplary embodiment of the invention;

FIGS. 22A-22C are each a schematic side view of guidewire placement devices according to another embodiment of the invention;

FIG. 23 is a schematic side view of an implant including a covering according to another embodiment of the invention;

FIGS. 24A-24B are schematic views of an implant, according to a further embodiment of the invention;

FIGS. 25A-25D are schematic views of implants, according to yet another embodiment of the invention; and

FIGS. 26 and 26A-26D are schematic views of an implant, according to a further embodiment of the invention.

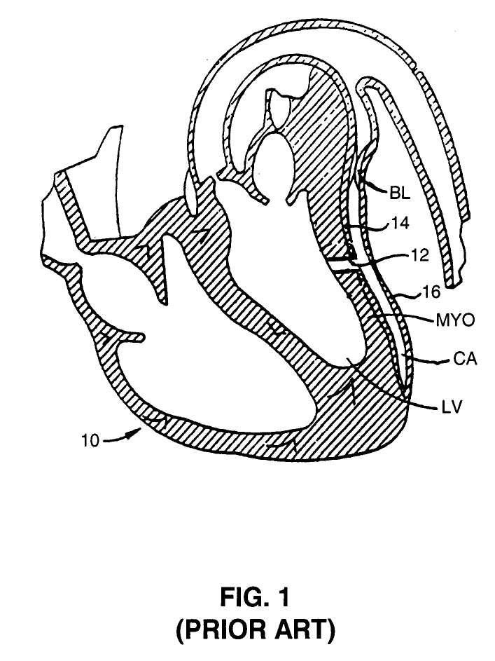

### DESCRIPTION OF EXEMPLARY EMBODIMENTS

Reference will now be made in detail to exemplary embodiments illustrated in the accompanying drawings. Wherever possible, the same reference numbers will be used throughout the drawings to refer to the same or like parts.

In exemplary embodiments, the present invention provides a multi-section implant for implanting in a body part, for example, in body tissue. An implant inserted into body tissue may have various uses, such as, for example, providing flow communication between two body parts, delivering drugs into a body part, or serving as a sensor, controller, or monitoring device. Exemplary embodiments of the present invention provide a heart implant, such as a stent, for example, having a two-section hinged structure. In addition, exemplary embodiments of the present invention provide a method of delivering and inserting such a two-section implant into the heart and a method of treating the heart using the implant to deliver blood from a chamber of the heart to a coronary vessel. Although certain exemplary embodiments described below relate to a two-section implant, it is contemplated that such an implant may have more than two sections. The number of sections and the location of each section relative to other sections may depend, for example, on the application for which the implant is being used and other factors influencing the number and relative positioning of the sections.

A heart wall implant according to exemplary embodiments of the invention may provide a direct blood flow passageway between a chamber of a heart, such as the left ventricle, and any coronary vessel, such as a coronary vein or a coronary artery, including for example a left anterior descending coronary artery, or between two coronary vessels, such as between a coronary artery and a coronary vein, for example. The devices and methods also encompass the use of an implant for delivering drugs into a body part or for serving as a sensor, controller, or monitoring device within the body.

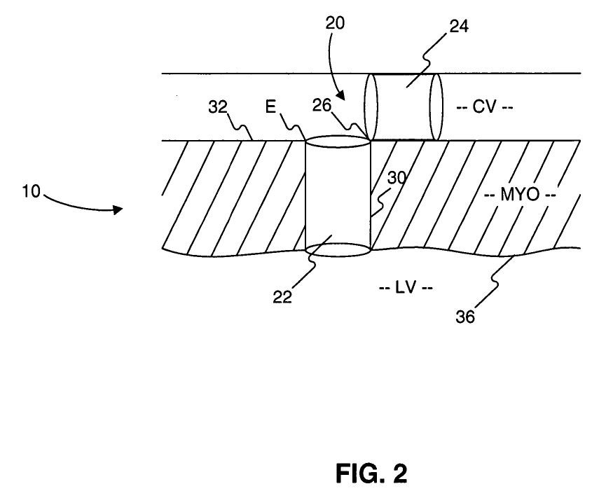

As shown in the exemplary embodiment of FIG. 2, a heart 10 has an implant in the form of a conduit or stent 20 in a heart wall MYO. The stent 20 includes two sections, a myocardial section 22 and a vessel section 24. Sections 22, 24 are connected by a hinged portion 26, permitting the sections 22, 24 to pivot relative to one another. Hinged portion 26, and other connectors set forth herein, may be made of any biocompatible material, for example, stainless steel. Hinged portion 26 may be an integral portion of one or both of sections 22, 24. Hinged portion 26 may alternatively be a portion completely separate from both sections 22, 24. If hinged portion 26 is not an integral portion of at least one of sections 22, 24, then it may be connected to that at least one section 22, 24, using any method or device disclosed herein.

Myocardial section 22 lies within a passage 30 in the myocardial wall between the posterior wall 32 of a coronary vessel CV (either a coronary artery or vein) to proximate the inner wall 36 of the left ventricle LV, potentially protruding into the heart chamber. Section 22 may lie in the heart wall MYO such that it is approximately flush with the floor (i.e., the posterior wall 32) of the coronary vessel CV. Myocardial section 22 may have a length in the range of approximately 10 mm to approximately 35 mm, for example, approximately 17 mm to approximately 28 mm, depending on the size of the patient's myocardium at the insertion site. That size may be determined by suitable measuring techniques, as described further below.

Vessel section 24 lies within the coronary vessel CV. Referring to FIG. 2, section 24 lies within the coronary vessel CV from the passage 30 to a location proximal to the passage. Vessel section 24 may have a length ranging from approximately 2 mm to approximately 20 mm, for example, approximately 5 mm to approximately 15 mm, depending on such factors as the diameter of the vessel and the location with respect to branch vessels. For instance, using a longer vessel section length may provide stability, while using a shorter vessel section length may enable side branches from the vessel to be avoided. Vessel section 24 may have an expanded diameter of approximately 2 mm to 6 mm, for example approximately 3 mm to approximately 5 mm, depending, for example, on the size of the vessel into which the vessel section is placed. Although in FIG. 2, section 24 is shown extending in a direction from the passage 30 to a location proximal to the passage 30, it should be understood that section 24 could extend in the opposite direction as well, for example, from the passage 30 to a location in the vessel distal the passage 30.

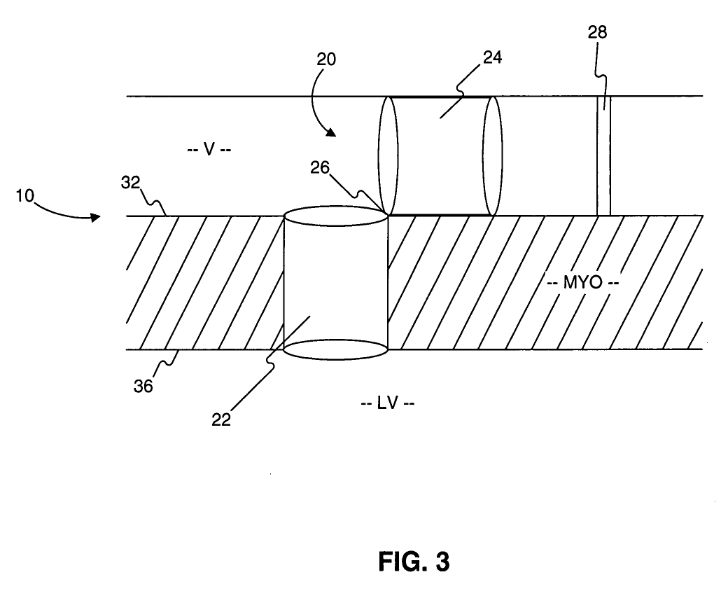

As used herein, the terms proximal and distal may refer to the direction that the delivery apparatus is introduced into the cardiovascular system as opposed to direction of blood flow. For example, in a percutaneous approach, a delivery apparatus would generally be introduced into the coronary vessels such that, whether it is introduced into the coronary artery via the coronary ostium or the coronary vein via the coronary sinus, the capillaries will be located distal to the delivery apparatus, regardless of the fact that the delivery apparatus may be advanced in the normal direction of flow in the coronary artery, and against the normal direction of flow in the coronary vein. Thus, the possibility of retrograde blood flow through the coronary vein, for example, as set forth in FIG. 3, would not change the proximal and distal designations. Accordingly, in the embodiments set forth herein, the vessel section of the implant will generally be placed proximal to the myocardial section and/or protection section (e.g., closer to the coronary ostium and/or coronary sinus), and the protection section of the implant will generally be placed distal to the myocardial section and/or vessel section (e.g., closer to the capillaries).

These designations are preferences and are not absolute, however. One of ordinary skill in the art would realize that the implant, including two or more of the vessel section, the myocardial section, and the protection section may be placed in any configuration relative to the coronary vessel and/or heart wall. For example, there may be some situations where the vessel section is placed distal to the myocardial section (e.g., closer to the capillaries). In addition, in some embodiments, it may be more advantageous to describe proximal and distal in terms of direction of blood flow.

As discussed above, the coronary vessel CV may be a coronary vein or a coronary artery, for example. When the implant is placed so as to flow blood from a heart chamber to a coronary vein, thereby causing retroperfusion in the vein so as to “arterialize” the vein, the implant may be used in conjunction with a venous blocking device. Such a venous blocking device may be placed in the lumen of the coronary vein relative to the vessel portion of the implant so as to at least partially occlude flow through the vein in the normal antegrade direction (e.g., toward the coronary sinus), thereby permitting the blood flow through the implant to flow in a retrograde direction through the coronary vein (e.g., toward the capillaries). FIG. 3 illustrates an implant in the form of a stent 20 having a myocardial section 22 and a vessel section 24 disposed within a coronary vein V. FIG. 3 further illustrates schematically an exemplary venous blocking device 28 positioned in the coronary vein V upstream, relative to the retrograde blood flow direction through the coronary vein V, of the stent 20. For further examples of venous blocking devices and particular applications of such devices when used in conjunction with an implant for flowing blood from a heart chamber to a coronary vein in order to cause retroperfusion, reference is made to U.S. Patent Application Publication No. 2005-0070993 A1, entitled “Methods of Retroperfusion and Related Devices,” the entire disclosure of which is incorporated herein by reference.

As discussed above, negative effects may result if the myocardial section 22 protrudes into the coronary vessel CV or is recessed within the heart wall MYO. For example, if section 22 protrudes too far into the lumen of the coronary vessel CV, blood flow through the coronary vessel CV (especially in the case of a coronary artery), as well as blood flow exiting from section 22 may become blocked, reducing the blood flow within the vessel. On the other hand, if section 22 is recessed within the heart wall MYO such that a space remains between section 22 and the posterior wall 32 of the coronary vessel CV, the space may become occluded with heart tissue, thereby hindering or preventing blood flow through section 22 and into the coronary vessel CV.

In addition to being properly positioned, the implant should not migrate from its position. In certain applications, it may be desirable to cover the inside, outside, or both, of a heart wall implant with a covering, such as, for example, a polymer covering such as an expanded polytetrafluoroethylene (ePTFE), polyurethane, collagen, or polyester fabric, for example, a tissue covering, or a metal covering, such as metal foil, for example. Such covering may help prevent the myocardial tissue from extruding into the lumen of the implant when a mesh-type stent is used. But this covering may reduce the frictional force that holds the implant in place and allow the implant to move away from an installed, desired position. In the case of a heart wall implant, the continuous, repetitive heart pumping action and variations in the blood flow may cause the implant in a heart wall to migrate from its designated location toward a left ventricle or a coronary vessel, for example. Thus, the implant may be vulnerable to migration for various different reasons, such as migration along the axis of the passageway, for example. As explained above, the migration of the implant may create undesirable risks, including but not limited to, stasis, occlusion of the passageway provided by the implant, vessel occlusion, embolization, and interference with the functioning of other body components, such as heart structures, for example.

Further, it is desirable to provide a mechanism by which to properly position the implant upon implantation.

Embodiments of the present invention may eliminate the above-mentioned risks and problems associated with improper initial positioning and migration by providing a multi-piece hinged implant, such as that shown in FIG. 2. The vessel section of the implant may help to anchor the implant, which may be in the form of a conduit or stent, in place in the heart wall so as to help prevent migration of the implant. In other words, the vessel section, such as vessel section 24, for example, may help prevent movement of the myocardial section 22 relative to the heart wall MYO either toward or away from the coronary vessel CV after implantation of the implant.

Additionally, a hinged implant, such as that illustrated in FIG. 2, may assist to position the implant properly during implantation. Such proper positioning may be achieved due to anchoring of the vessel segment of the implant, which, due to its connection with the myocardial segment, may in turn appropriately position the myocardial segment.

The stent 20 in the exemplary embodiment of FIG. 2 may be a non-mesh stent, a mesh stent, a coil type stent, or other type of stent, for example. Some exemplary embodiments of suitable coil type stents are provided in U.S. application Ser. No. 09/917,655, entitled “Myocardial Stents and Related Methods of Providing Direct Blood Flow from a Heart Chamber to a Coronary Vessel,” filed Jul. 31, 2001 (published as U.S. Patent Application Publication No. US 2002-0032478 A1 on Mar. 14, 2002), the entire disclosure of which is incorporated by reference herein. Also, stent 20 may be provided with a covering and/or a coating. For example, the stent 20 may be provided with a polymeric covering, for example an ePTFE covering, on its inner and/or outer surfaces. Additionally, a coating, such as a heparin coating, for example, may be provided on the covering on the inner and/or the outer surface.

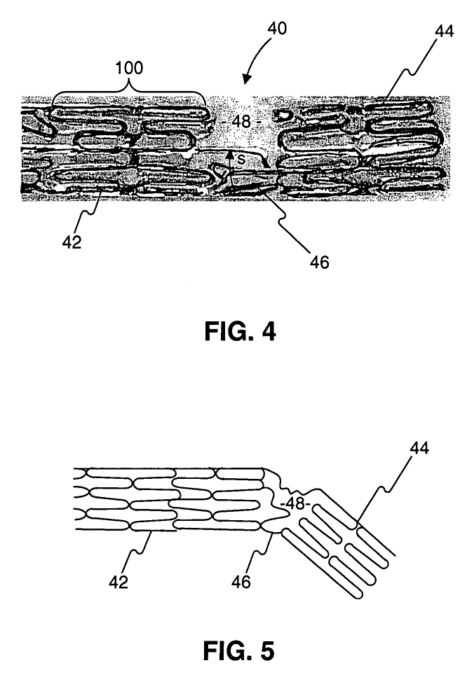

FIG. 4 shows an exemplary embodiment of a stent 40 in an undeployed straight configuration, and FIG. 5 shows stent 40 in a hinged, deployed configuration. In the exemplary embodiment of FIG. 4, stent 40 comprises a plurality of stent cells. As an example, a stent cell may be defined as one of the repeating annular segments of a stent that extends along the length of the stent and that forms a portion of the stent structure. For example, FIG. 4 shows a plurality of stent cells 100. In an exemplary embodiment, vessel section 44 may extend along a length defined by one or more stent cells. By way of example only, vessel section 44 of the stent 40 shown in FIG. 4 extends one stent cell length.

In the example shown in FIG. 4, a hinged portion 46 connects vessel section 44 to myocardial section 42 so as to permit those sections to bend relative to each other. The hinged portion 46 is integrally formed with sections 42, 44. In this and like examples, hinged portion 46 comprises one or more wires (e.g., struts) of a stent cell. In the exemplary embodiment shown in FIG. 4, hinged portion 46 extends the length of about one half of a stent cell and comprises only a circumferential segment S of that half stent cell. In other words, a portion of the half stent cell around the circumference of the stent 40 is missing so as to form the flexible hinged portion 46. This missing portion is sufficient to leave an opening 48 between the myocardial section 42 and the vessel section 44 that allows the myocardial section 42 and the vessel section 44 to articulate relative to each other. The missing portion around the circumference of the stent thus leaves a remaining circumferential segment that forms the hinged portion 46. The missing circumferential portion may range, for example, from approximately 270 degrees to approximately 350 degrees, and the circumferential segment S that remains may range from approximately 10 degrees to approximately 90 degrees.

In the exemplary embodiment shown in FIG. 4, approximately 300 degrees around the circumference of the half stent cell is missing, thereby leaving a circumferential segment S of approximately 60 degrees. The length of the hinged portion 46 may range from approximately 0.25 mm to approximately 2 mm, for example, the length of the hinged portion 46 may be approximately 0.5 mm. The hinged stent 40 that is formed by the removal of a circumferential segment of at least part of a stent cell thereby forming opening 48 differs from other stent structures in which a side opening, for example, a circular side opening that simply permits fluid flow through the side of a stent or permits another stent to be inserted therein, is formed and does not permit relative articulation of stent sections on either side of the side opening.

Myocardial section 42 of stent 40, only a portion of which is shown in FIG. 4, comprises a plurality of stent cells 100 totaling a length approximating the thickness of the myocardium. For example, the myocardial section 42 may have a length ranging from approximately 10 mm to approximately 35 mm, as discussed above, and may be formed of approximately 2 stent cells to approximately 12 stent cells.

A stent according to embodiments of the invention may be elastic or have expandable and/or collapsible structures. The collapsed stent may facilitate the delivery of the implant into the heart wall MYO by providing a smaller structure during delivery.

An implant (e.g., stent) according to exemplary embodiments of the invention described herein can be made from a biocompatible metal material, such as, for example, stainless steel, nickel (Ni) alloys, titanium (Ti) alloys, nickel-titanium alloys, cobalt-based alloys, titanium, tantalum, and other similar suitable metal materials. Examples of nickel, titanium, or nickel-titanium alloys may include NiTi shape memory alloys and NiTi super elastic alloys. Alternatively, the implant can be made from a biocompatible polymer. Examples of biocompatible polymers include polytetrafluoroethylenes (PTFEs), polyetheretherketones (PEEKs), polyesters, polyurethanes, polyamides, ePTFEs, and other similar suitable polymers. Further, the implant may be formed of a bioabsorbable material, for example a bioabsorbable metal or polymer. Examples of suitable bioabsorbable polymers include polylactic acid (PLA), polycaprolactone (PC), and polyglycolic acid (PGA), for example. It is contemplated that any combination of these various materials may also be used to form an implant according to the invention. For example, the hinged portion of the implant could be made of a different material than the remaining portions of the implant. As another example, the various implant sections may be made of differing materials.

Embodiments of the present invention may include various multiple-section hinged implants and are not limited to the embodiments discussed above. For example, the implants need not be in the form of stents, but may be in the form of other types of implants for which it may be desirable to have two or more sections that can pivot relative to each other. Further, the hinged portion or portions joining the various sections of the implant could be configured so that there is a maximum angle at which the various sections may bend relative to each other. As an example, the hinged portion may be configured to self-deploy to assume a desired angle between the joined sections. Additionally, as discussed in more detail below, the implant may be formed of three sections so as to form a “T” shaped configuration, with two of the sections configured to articulate relative to a third section. Further, the entire implant or sections, such as the myocardial and/or vessel section of the implant, may be self-expandable.

The following paragraphs describe exemplary embodiments of delivery systems and methods for inserting a hinged implant into a body part. The following methods are described in connection with delivering and positioning stent 40 into a passage between a left ventricle and a coronary vessel.

Generally, the delivery of an implant may be accomplished by an implant delivery system. An implant delivery system may provide one or more functions, such as providing access to an insertion site or a location near the insertion site, providing a passageway for insertion, delivering an implant into a body, and inserting the implant into the body part, for example. As an exemplary embodiment, one or more catheters may be inserted percutaneously or surgically into a body. The catheter may be inserted into the body with or without a guidewire that guides the entry of the catheter during the insertion process.

In an exemplary embodiment of obtaining access to a heart wall under a percutaneous approach, the catheter may be inserted through a femoral vessel and advanced in the patient's vasculature to a coronary vessel. Alternatively, the catheter may obtain access to the vasculature under an open-chest or other surgical approaches. For example, the catheter may be inserted through the anterior wall and posterior wall of a coronary vessel and then into the heart wall.

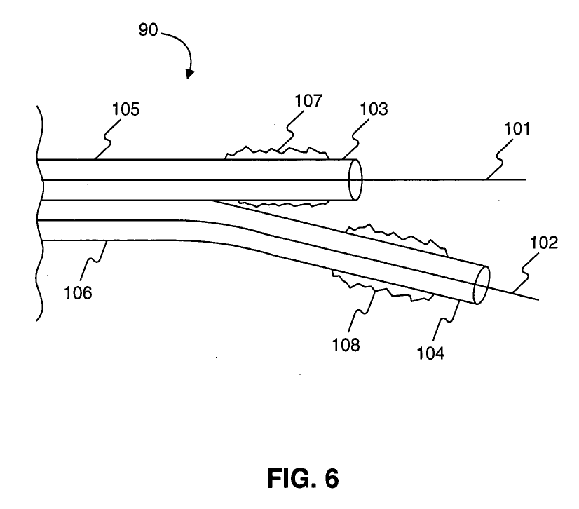

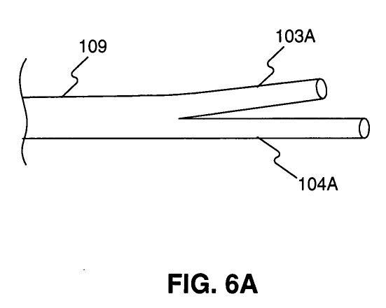

FIG. 6 schematically shows an exemplary embodiment of a delivery system according to the invention. The system includes a catheter delivery system that includes two guidewires 101, 102. The guidewires 101 and 102 respectively exit through the lumens defined by two branched shafts 103, 104 disposed at a distal end portion of a catheter 90. The branched shafts 103, 104 may be disposed substantially side-by-side. A portion of one shaft 104 may extend distally past the distal end of the other shaft 103 to some extent. The branched shafts 103, 104 are configured so as to move independently from one another, as will be explained in more detail below.

The catheter 90 may have several different forms. For example, the catheter 90 may comprise dual side-by-side catheter shafts 105, 106 secured together at least near the proximal end portion of the catheter 90. Such securing of the catheter shafts 105, 106 may assist in reducing twisting of the system. Alternatively, as shown in FIG. 6A, a catheter could have a single shaft 109 at the proximal end portion that branches into two shafts 103A, 104A at the distal end portion. Other embodiments of catheters having branched shafts at an end thereof also are envisioned and are considered within the scope of this invention.

Each of the branched shafts 103,104 carries a balloon 107, 108, shown in a deflated state in FIG. 6. The catheter 90 may have one or more ports (not shown) disposed at a proximal end thereof to supply fluid to inflate the balloons 107, 108. Balloons 107, 108 may be separately inflatable via different ports each in respective flow communication with balloons 107,108, or may be configured so as to inflate via a single port in flow communication with both balloons 107, 108. In addition, the proximal end of a catheter delivery system according to exemplary embodiments of the invention may include any suitable actuation mechanism (not shown) for supplying inflation fluids to the catheter and the balloons. As shown in the exemplary embodiment of FIG. 6, the balloon 108 may be mounted distal to the balloon 107, although it is envisioned that at least portions of the balloon 107 and 108 also may overlap. Alternatively, the balloon 107 may be mounted distal to the balloon 108.

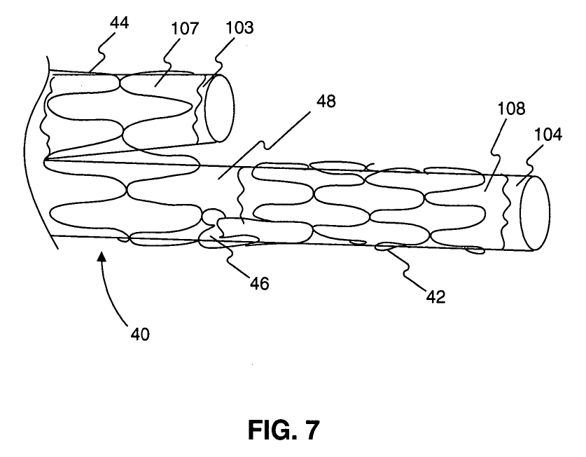

FIG. 7 illustrates the implant of FIGS. 4 and 5 carried by the catheter 90 of FIG. 6 according to an exemplary configuration. The proximal end portion of the delivery system is not shown, but extends further to the left side of FIG. 7. As shown, the shaft 103 carrying balloon 107 is inserted through the vessel section 44 of the stent 40 and exits from the opening 48 of the stent 40. The shaft 104 carrying balloon 108 is inserted through the vessel section 44 and the myocardial section 42, exiting through the end of the myocardial section 42 furthest from the vessel section 44. In the exemplary embodiment of FIG. 7, the portion of shaft 104 that extends through vessel section 44 does not carry the balloon 108. Rather, the balloon 108 extends only through the myocardial section 42 of the stent 40.

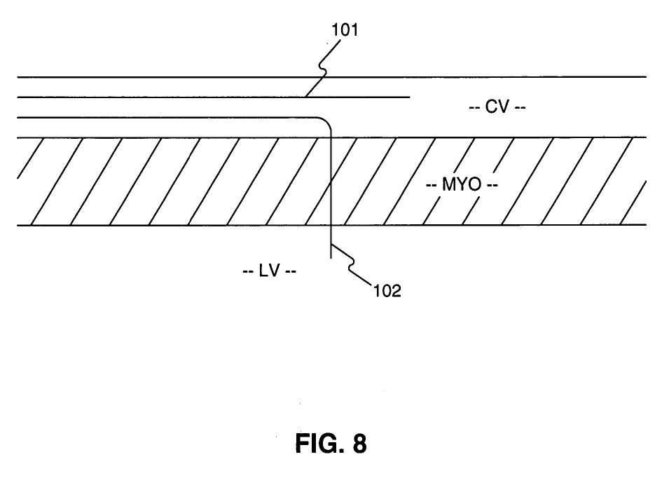

Using the delivery system of FIGS. 6 and 7 to deliver the stent 40 to a heart wall between the heart chamber and a coronary vessel, guidewires 101, 102 are inserted down the coronary vessel CV, as shown in FIG. 8. Upon reaching the location of the myocardium MYO at which it is desired to position the stent 40, guidewire 102 is tracked into the myocardium MYO. The guidewire 102 may be tracked into the myocardium MYO using any suitable method for orienting the guidewire, including but not limited to, using puncturing techniques and imaging techniques, for example. Guidewire 101 is advanced down the coronary vessel CV to a position distal to the location in the heart wall MYO at which the guidewire 102 is inserted.

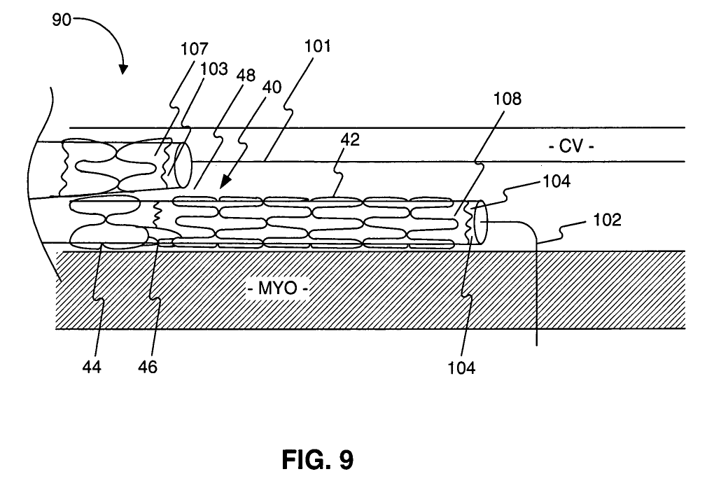

Once the guidewires 101,102 are in position, as show in FIG. 8, the catheter 90 carrying the stent 40, as shown in FIG. 9, may then be advanced over the two guidewires 101,102 from their proximal ends. That is, the shaft 103 carrying vessel section 44 may be advanced over and along guidewire 101, and the shaft 104 carrying both the vessel section 44 and myocardial section 42 may be advanced over and along guidewire 102. As the catheter 90 carrying the stent 40 is advanced along the guidewires 101,102, through the coronary vessel CV, eventually, the shaft 104 carrying myocardial section 42 will be advanced along guidewire 102 and into the myocardium MYO. As the myocardial section 42 is advanced along guidewire 102 and into the myocardium MYO, the vessel section 44 bends relative to-the myocardial section 42 at hinge 46 and acts as a “stop” to prevent the vessel section 44 from being advanced into the myocardium MYO. In particular, because the vessel section 44 is advanced over the guidewire 101 which extends down the vessel CV, and not into the myocardium MYO, the vessel section 44 is substantially prevented from being advanced into the myocardium MYO. In this way, the stent 40 is deployed with the myocardial section 42 disposed in the myocardium MYO and the vessel section 44 remaining in the vessel CV at a location proximal to the location of the myocardial section 42 in the direction of insertion down the vessel CV, as shown in FIG. 12. Delivering the implant using this approach allows the physician to receive tactile feedback at the site where the wires bifurcate so as to determine proper positioning of the implant.

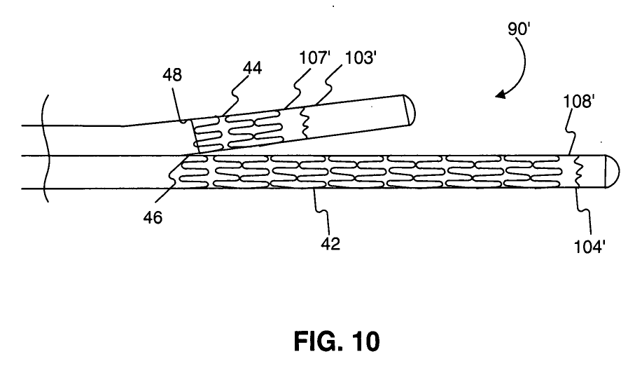

FIG. 10 illustrates the implant of FIGS. 4 and 5 carried by a branched catheter delivery system 90′ according to another exemplary configuration. Only part of the proximal portion of the delivery system is shown; the remaining proximal portion extends to the left side of the FIG. 10 and is not shown. Catheter 90′ and catheter 90 may have similar configurations. However, respective sizes of the catheter shafts may differ as needed depending on the size of the stent, the method of delivery employed, and other similar factors.

As shown, the shaft 103′ carrying balloon 107′ is inserted through the opening 48 and the vessel section 44 of the stent 40. The shaft 104′ carrying a balloon 108′ is inserted through the opening 48 and the myocardial section 42, exiting through the end of the myocardial section 42 furthest from the vessel section 44. The loading of the stent 40 onto the catheter 90′ differs from that of FIG. 7 in that the shaft 103′ of the catheter 90′ exits through the open end of vessel section 44 and not through the opening 48. Further, the shaft 104′ extends through only the myocardial section 42, as opposed to through both the vessel section 44 and the myocardial section 42 in the embodiment of FIG. 7. In this way, the catheter 90′ is inserted through the stent 40 such that the vessel section 44 is folded over the myocardial section 42 at the hinge 46.

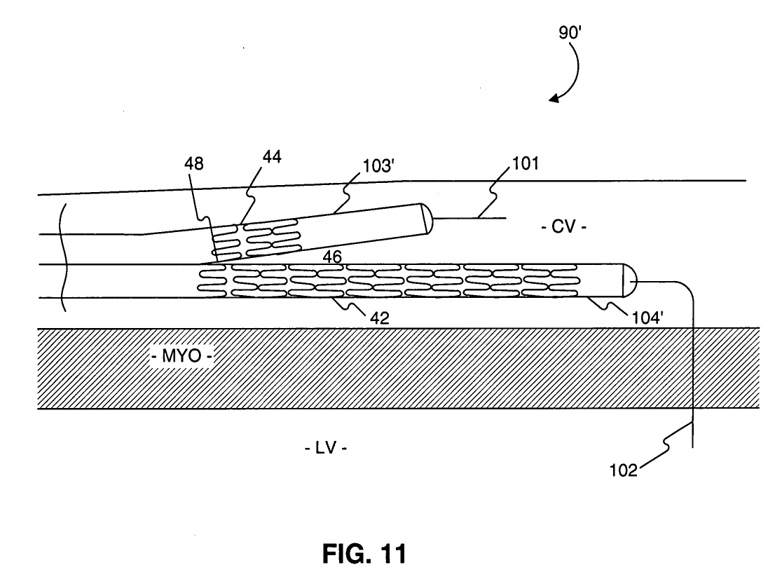

Using the delivery system of FIG. 10 to deliver the stent 40 to a heart wall between the heart chamber and coronary vessel, guidewires 101, 102 are inserted down the coronary vessel CV, as shown and described above with respect to FIG. 8. Upon reaching the location of the myocardium MYO at which it is desired to position the stent 40, guidewire 102 is tracked into the myocardium MYO, for example, using suitable orienting, imaging, and/or puncturing techniques. The catheter 90′ carrying the stent 40 may then be advanced over the two guidewires 101, 102 from their proximal ends, as shown in FIG. 11. That is, the shaft 103′ carrying vessel section 44 may be advanced over and along guidewire 101 and the shaft 104′ carrying myocardial section 42 may be advanced over and along guidewire 102.

As the catheter 90′ carrying the stent 40 is advanced along the guidewires 101, 102, through the coronary vessel CV, eventually, the shaft 104′ carrying myocardial section 42 will be advanced along guidewire 102 and into the myocardium MYO. As the myocardial section 42 is advanced along guidewire 102 and into the myocardium MYO, the vessel section 44 acts as a “stop” to prevent the vessel section 44 from being advanced into the myocardium MYO and also provides tactile feedback to the physician to ensure proper positioning of the stent 40. In particular, because the vessel section 44 is advanced over the guidewire 101 which extends down the vessel CV, and not into the myocardium MYO, the vessel section 44 is substantially prevented from being advanced into the myocardium MYO. In this way, the stent 40 is deployed with the myocardial section 42 disposed in the myocardium MYO and the vessel section 44 remaining in the vessel CV at a location distal to the location of the myocardial section 42 in the direction of insertion down the vessel CV (i.e., the stent 40 is disposed in the position shown in FIG. 2).

In another exemplary embodiment, a self-expandable implant may be used and a sheath delivery mechanism may be used to deliver the implant. Such a sheath delivery mechanism may have a similar structure to the shafts of the catheter-based systems described above with respect to FIGS. 6 and 6A and the delivery of such a sheath delivery mechanism may be similar to that described with respect to FIGS. 8-11. However, instead of the implant being supported on balloons external to the shafts, the implants could be inserted into the sheath shafts and the shafts could be retracted once an implant section was in an appropriate position, causing the implant section to expand and be implanted. Those skilled in the art would understand how such a sheath delivery mechanism could be used to deliver the hinged implants described herein.

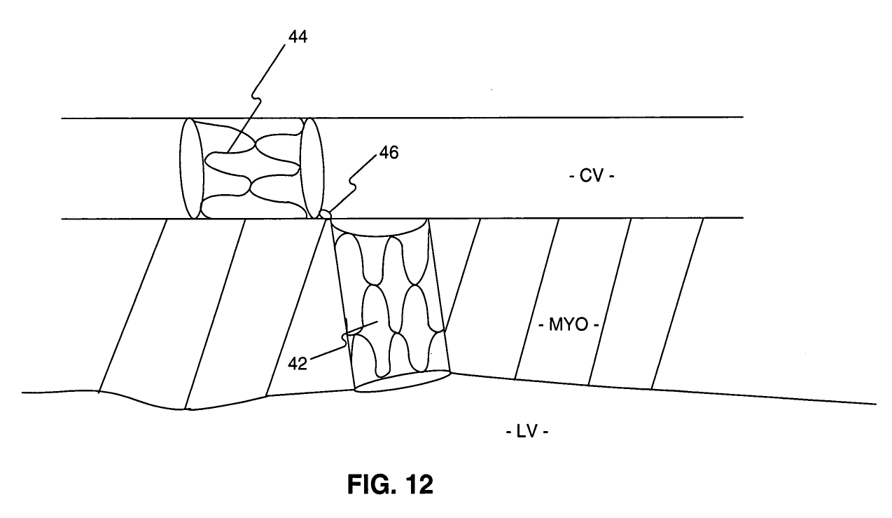

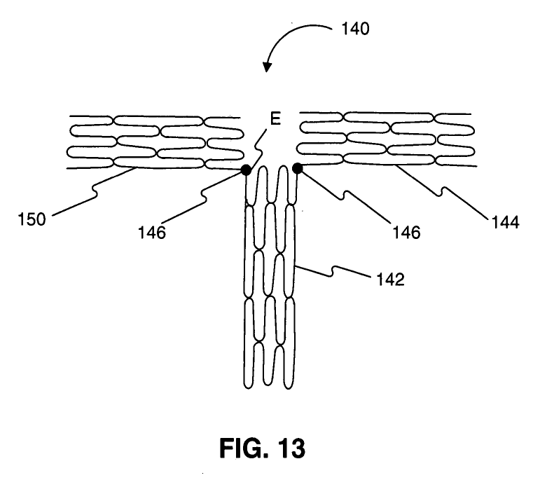

FIG. 13 shows an exemplary embodiment of a stent 140, which has three sections: a vessel section 144, a myocardial section 142, and a protection section 150. The vessel section 144 and the myocardial section 142 are substantially similar to the vessel section 44 and the myocardial section 42 described above. The protection section 150 is configured to protect the portion (e.g., edge E shown in FIGS. 2 and 13) of the myocardial section 142 located opposite to the portion which the vessel section 144 is joined. In particular, the edge E may be positioned too high relative to the myocardium when the stent is implanted such that it protrudes into the vessel lumen, which may disrupt blood flow. Alternatively, the edge E may be positioned too low such that myocardial tissue and/or the vessel floor may cover the edge E and encroach into the opening of the myocardial portion 142 and hinder blood flow therethrough. Further, the protection section 150 may be designed so as to maintain distal blood flow through the vessel.

The vessel section 144 and/or the protection section 150 may be connected to the myocardial section 142 in any suitable combination using any suitable method and/or apparatus set forth herein. For example, two or more of myocardial section 142, vessel section 144, and/or protection section 150 may be integrally connected to each other and/or may be connected to each other via one or more hinges 146 that are substantially similar to the hinge 46 described above. The hinge 146, or any other connector set forth herein, may be made of any suitable biocompatible material, for example, stainless steel. Alternatively, the vessel section 144 and/or protection section 150 may be connected to the myocardial section 142 via other connection mechanisms, such as, for example, tethers or a weld.

For example, myocardial section 142 and vessel section 144 may be integrally connected to each other via hinge 146 (e.g., hinge 146 may be machined from the same piece of material as myocardial section 142 and vessel section 144), and then protection section 150 may be a separate section connected to one of myocardial section 142 and vessel section 144 via a tether or weld. In another example, myocardial section 142, vessel section 144, and protection section 150 may each be separate sections connected by welds and/or discrete tethers. In a further example, myocardial section 142 may be connected to vessel section 144 via a first weld or tether, and protection section 150 may be connected to myocardial section 142 via a second weld or tether. In another example, two or more of myocardial section 142, vessel section 144, and protection section 150 may be connected to each other via both hinge 146 and a tether.

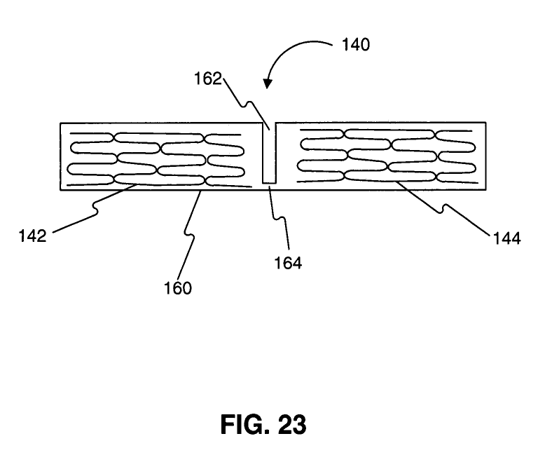

When deployed, the stent 140 may have a substantially T-shaped configuration. A tether, or any other connector set forth herein, may be made of any suitable biocompatible material, for example, PTFE. For example, as shown in FIG. 23, stent 140 may include two separate sections 142, 144 covered by a covering 160, such as a PTFE covering. Covering 160 may be a missing a circumferential portion 162 on the portion of covering 160 disposed between the two sections. Thus, the remaining portion 164 of covering 160 may act as a tether, or hinge, to connect the two sections. Section 150 may be connected to one or both of sections 142, 144 in a similar fashion.

The stent 140, including the vessel section 144, myocardial section 142, and protection section 150, may be fabricated in a variety of ways. For example, the vessel section 144, myocardial section 142, and protection section 150 may be fabricated from the same section of tubing. Alternatively, the vessel and protection sections 144, 150 may be fabricated from the same tubing and then connected to the myocardial section 142 via, for example, welding, tethers, or other connection mechanisms. In another example, the vessel section 144 and myocardial section 142 may be connected to each other (e.g., formed from the same tubing), and then the protection section 150 may be connected to the myocardial section 142. In another exemplary embodiment, the protection section 150 could be separated (e.g., not connected) to the hinged stent made of the myocardial and vessel sections 142, 144. In this configuration, the protection section 150 could be delivered to the vessel before or after the hinged stent has been implanted. The protection section 150 may then be connected to one of the myocardial and vessel sections 142, 144.

Guidewires 201, 202 may be deployed in the coronary vessel and/or the myocardium using any method known in the art, for example, percutaneously.

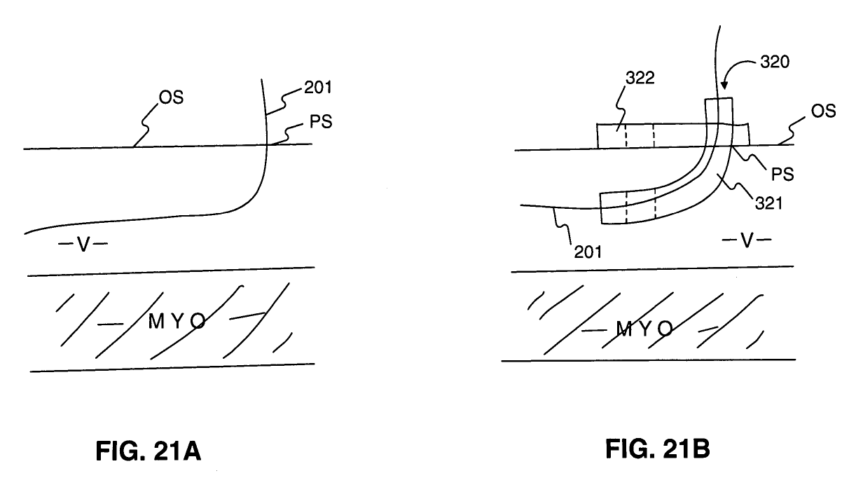

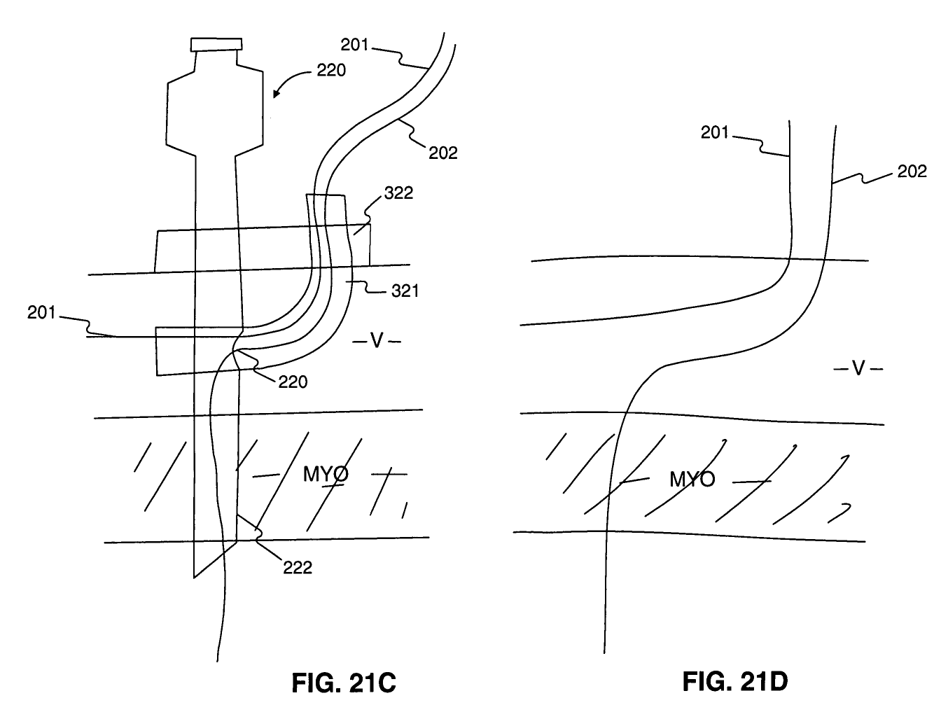

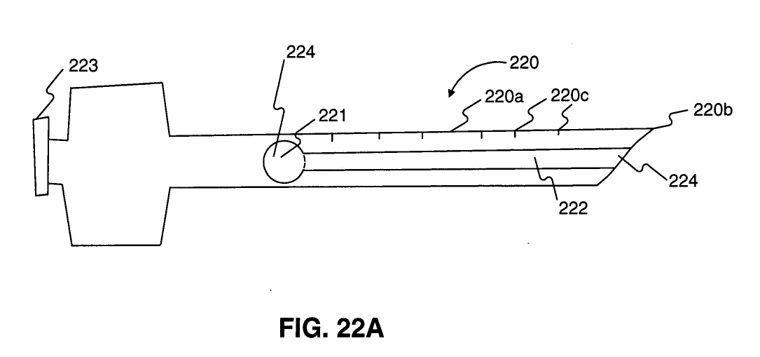

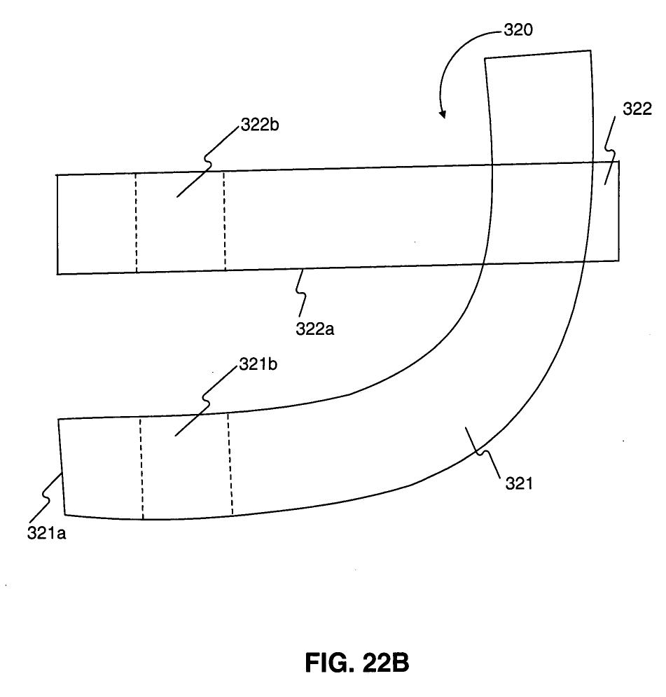

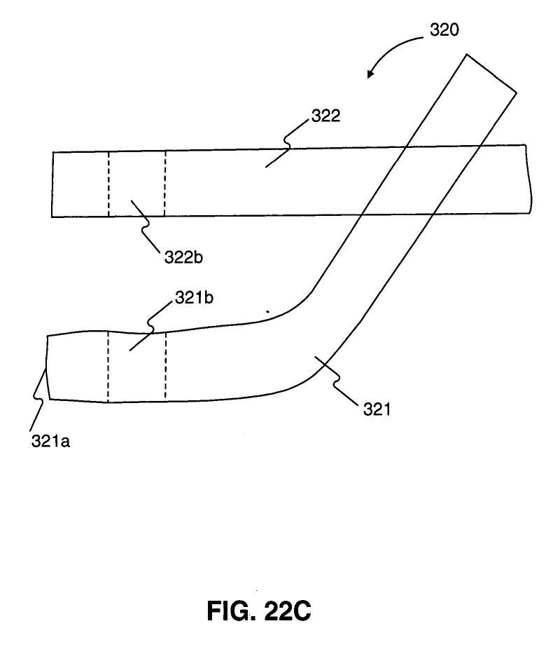

FIGS. 21A-21D, and 22A-22C depict exemplary apparatuses and methods for deploying guidewires 201, 202 using a surgical approach. As shown in FIG. 21A, a first guidewire 201 may be advanced through a wall of the coronary vessel at a puncture site PS such that the first guidewire 201 is now disposed in the coronary vessel V. The first guidewire 201 may then be advanced through the coronary vessel V, for example, until a distal end of first guidewire 201 is disposed past a site in the myocardium MYO at which stent 140, 240 will be deployed. First guidewire 201 may be deployed with the assistance of any device, for example, an angiocatheter.

A first portion 321 of a guide tool 320 may then be advanced over first guidewire 201. As shown in FIGS. 22B and 22C, first portion 321 may be curved and may include a channel along its entire length to receive guidewire 201. First portion 321 may define a guide 321b (e.g., a hole) near an end 321a. Tool 320 includes a second portion 322 connected to first portion 321. Second portion 322 may form an angle with respect to first portion 321, for example, about 90 degrees as shown in FIG. 22B or about 120 degrees as shown in FIG. 22C. Second portion 322 may define a guide 322b (e.g., a hole) substantially aligned with and/or coaxial with guide 321b. Guides 321b, 322b may be configured to accept a 19 Ga needle and/or have a diameter between about 1 mm and about 2 mm. Any portion of guide tool 320 may be made out of any suitable material, for example, nitinol, stainless steel, and/or a suitable polymer.

End 321a of first portion 321 may be placed through puncture site PS and into coronary vessel V. First portion 321 may be advanced into coronary vessel V until a surface 322a of second portion 322 is substantially flush with an outer surface OS of the coronary vessel V, for example, as shown in FIG. 21B.

A sharp end 220b of a puncture tool 220 may then be advanced through guides 321b, 322b (e.g., holes) disposed on first and second portions 210, 220 on guide tool 320, and into the myocardium, for example, as shown in FIG. 21C. Puncture tool 220 may include an outer surface 220a substantially shaped like a needle with sharp distal end 220b. Outer surface 220a may define a hole 221 and a slit 222 that extends from hole 221 to distal end 220b. Hole 221 and slit 222 may only be disposed on one portion of outer surface 220a (e.g., hole 221 and slit 222 may not extend through puncture tool 220). An inner surface 224 of puncture tool 220 opposite hole 221 and slit 222 may be solid, and may be configured, for example, to deflect a guidewire placed through hole 221 and/or slit 222. The guidewire may be deflected by inner surface 224 towards distal end 220a. Puncture tool 220 may be used to measure the thickness of the myocardium, for example, via markers 220c disposed on the outer surface 220a of puncture tool 220. Puncture tool 220 may be rotated such that hole 221 is in flow communication with an inner lumen of first portion 321 in the direction of puncture site PS, for example, as shown in FIG. 21C. A second guidewire 202 may then be advanced through the inner lumen of first portion 321 and through hole 221 into a lumen of tool 220. An inner surface 224 of tool 220 may then deflect second guidewire 202 toward distal end 220b, and then out distal end 220b. Additionally or alternatively, an end of second guidewire 202 may be configured so as to facilitate movement of second guidewire 202 toward distal end 220b, for example, by being bent, rotated, and/or positioned through hole 221 so as to point substantially towards distal end 220b. Thus, second guidewire 202 may be placed through the myocardium and into the heart chamber (e.g., left ventricle).

Once second guidewire 202 has been deployed, puncture tool 220 may be removed from guides 321b, 322b and second guidewire 202 may remain in the coronary vessel V and the myocardium because second guidewire 202 may exit the lumen of puncture tool 220 via slit 222. Accordingly, guidewires 201, 202 may be disposed in coronary vessel V and myocardium MYO, for example, as shown in FIG. 21D.

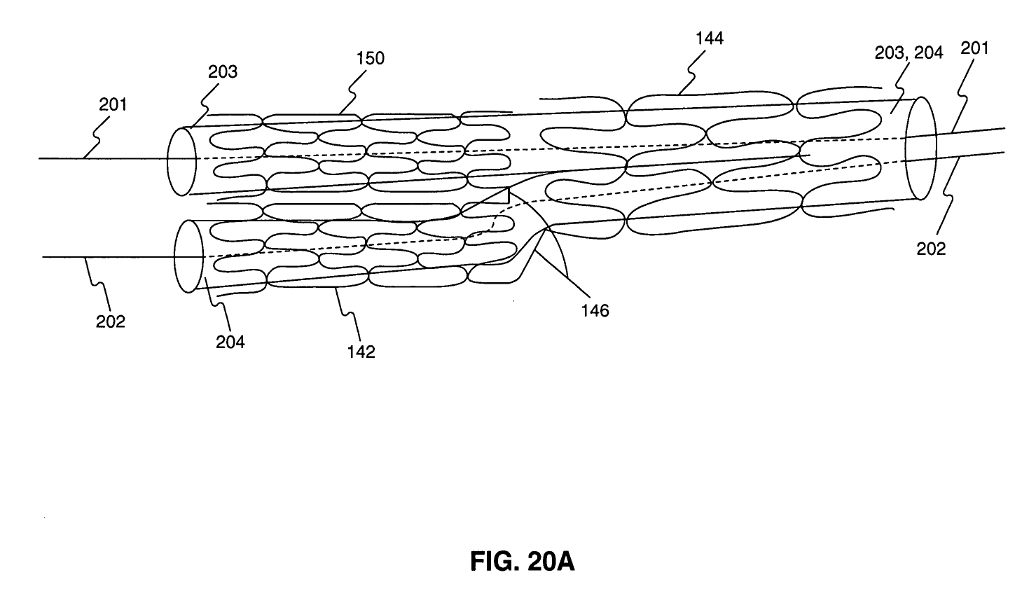

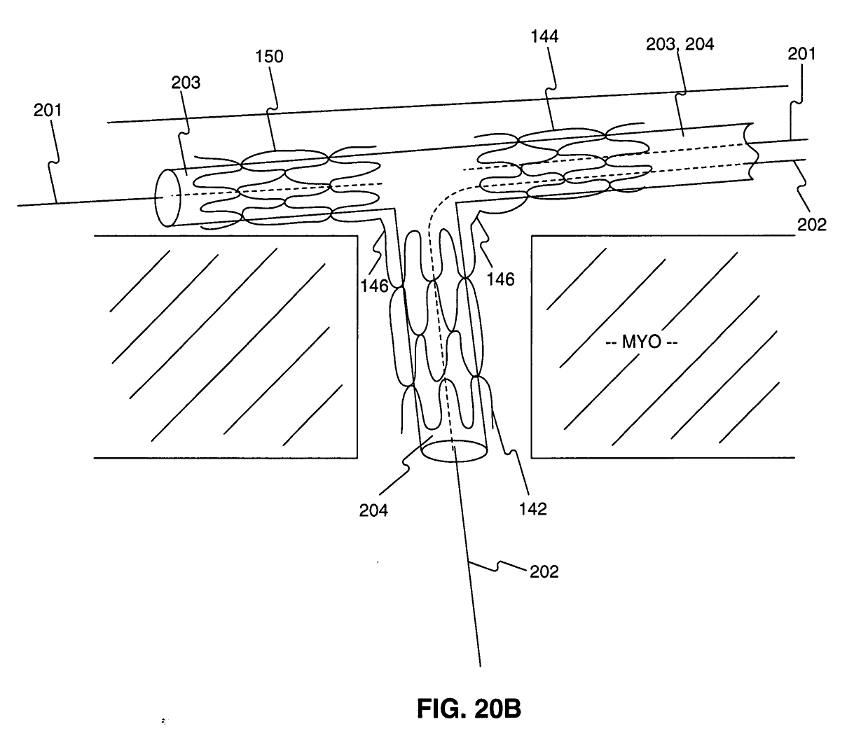

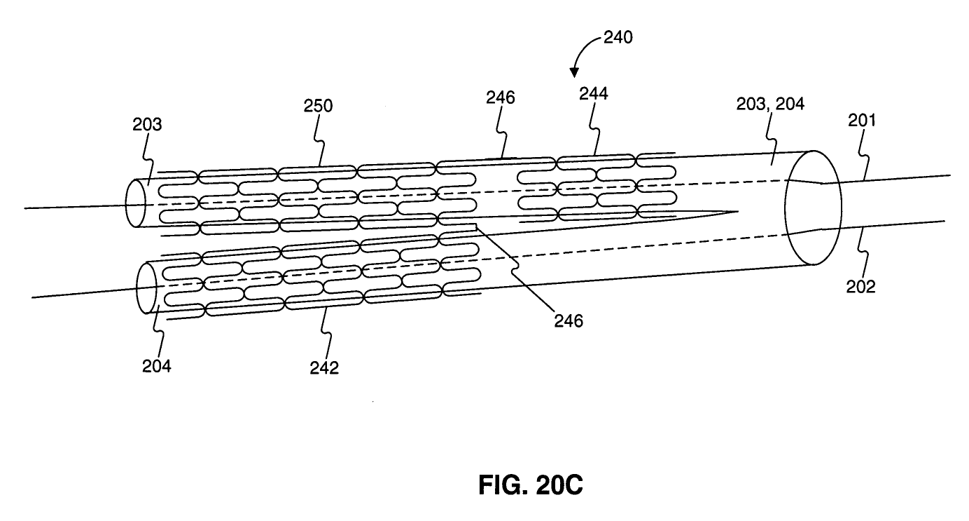

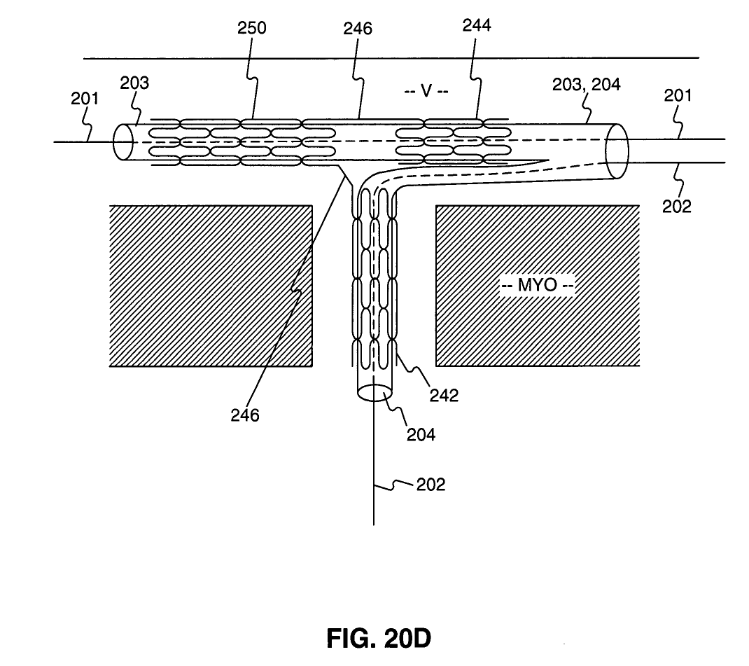

Once guidewires 201, 202 have been placed in the coronary vessel and/or heart wall, stent 140, 240 may be deployed, for example, as shown in FIGS. 20A-20D.

In one embodiment shown in FIGS. 20A and 20B, a first catheter 203 guided by first guidewire 201 may be placed through the protection section 150 and vessel section 144 of stent 140, and a second catheter 204 guided by second guidewire 202 may be placed through the myocardial section 142 and the vessel section 144 of stent 140. In alternate embodiments, the second catheter 204 guided by the second guidewire 202 may be placed through the myocardial section 142 and the protection section 150. Once the catheters 203, 204 are deployed in their respective sections 142, 144, 150, the myocardial section 142 may be pivoted away from the vessel section 144 and toward the protection section 150, as shown in FIG. 20A.

Once stent 140 is disposed on first and second catheters 203, 204, stent 140 may be advanced along first and second guidewires 201, 202. The stent 140 may then be advanced into the coronary vessel V along the guidewires 201, 202 with the myocardial section 142 and the protection section 150. Once the stent 140 reaches the appropriate site in the coronary vessel and/or myocardium, the myocardial portion 142 may be advanced through the myocardium via the second guidewire 202 (e.g., pivoted toward the vessel section 144 and/or away from protection section 150) and the protection section 150 may be advanced further down the coronary vessel via the first guidewire 201. Thus, stent 140 may assume the configuration shown in FIG. 20B. Once so positioned, one or more of the stent sections 142, 144, 150 may be expanded and the stent 140 may be deployed. For example, catheter 203, 204 may include expandable balloons positioned inside a lumen defined by one or more of stent sections 142, 144, 150, that may be expanded through any known method. Alternatively, stent sections 142, 144, 150 may be self-expandable, and any suitable means, such as a sheath over catheters 203, 204, may be used to-unconstrain stent sections 142, 144, 150 to permit self-expansion. Once the stent sections are expanded, catheters 203, 204 and guidewires 201, 202 may be removed.

In another embodiment shown in FIGS. 20C and 20D, a first catheter 203 guided by a first guidewire 201 may be placed through the protection section 250 and vessel section 244 of stent 240, and a second catheter 204 guided by a second guidewire 202 may be placed only through the myocardial section 242. Once the catheters 203, 204 are deployed in their respective sections 242, 244, 250, the myocardial section 242 may be pivoted away from the protection section 250 and toward the vessel section 244, as shown in FIG. 20C.

Once stent 240 is disposed on first and second catheters 203, 204, stent 240 may be advanced along first and second guidewires 201, 202. The stent 240 may then be advanced into the coronary vessel V along the guidewires 201, 202 with the myocardial section 242 and the protection section 250. Once the stent 240 reaches the appropriate site in the coronary vessel and/or myocardium, the myocardial portion 242 may be advanced through the myocardium via the second guidewire 202 (e.g., pivoted toward the vessel section 244 and/or away from protection section 250) and the protection section 250 may be advanced further down the coronary vessel via the first guidewire 201. Thus, stent 240 may assume the configuration shown in FIG. 20D. Once so positioned, one or more of the stent sections 242, 244, 250 may be expanded and the stent 240 may be deployed, for example, as described above.

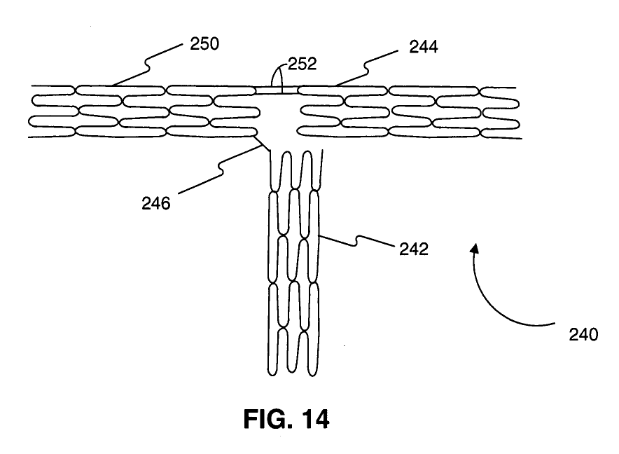

The various delivery techniques and tools described with reference to FIGS. 6A-11 also may be used to deliver the implants of FIGS. 13 or 14.

Embodiments of a stent according to the present invention may have a variety of alternate configurations. For example, a protection section 250 may be a stent-like slotted tubing structure as shown in FIG. 14. A portion of an end of the protection section 250 may be connected to a portion of an end of a vessel section 244, for example, via one or more connectors 252. A portion of an end of a myocardial portion 242 may be connected to a portion of an end of at least one of the vessel section 244 and the protection section 250 via a welded portion 246.

The stent 240, including the vessel section 244, myocardial section 242, and protection section 250, may be fabricated in a variety of ways. For example, the vessel section 244 and protection section 250 may be fabricated separately and then connected. The myocardial section 242 may be connected to the vessel section 244 prior or subsequent to connection of the vessel section 244 and the protection section 250. In another example, the vessel section 244 and protection section 250 may be fabricated from the same section of tubing with portions of the tubing being removed. The myocardial section 242 may then be connected to the vessel section 244.

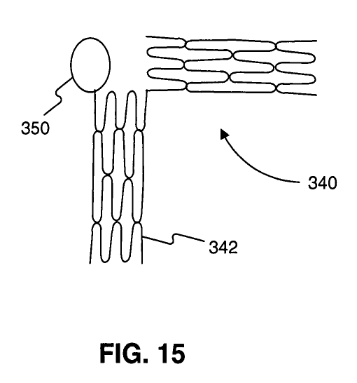

In another example, a protection section 350 may be a metal ring connected to a myocardial portion 342, as shown in FIG. 15. The protection section 350 may be connected to the myocardial portion 342 prior or subsequent to deployment of the stent 340. To deploy the protection section 350, the protection section 350 may be crimped onto a balloon catheter, and then once properly positioned, the balloon may be expanded so as to deploy the protection section 350 in the coronary vessel.

In a further example, the protection section may be an extension of the vessel section with an opening for the myocardial section to be placed therethrough.

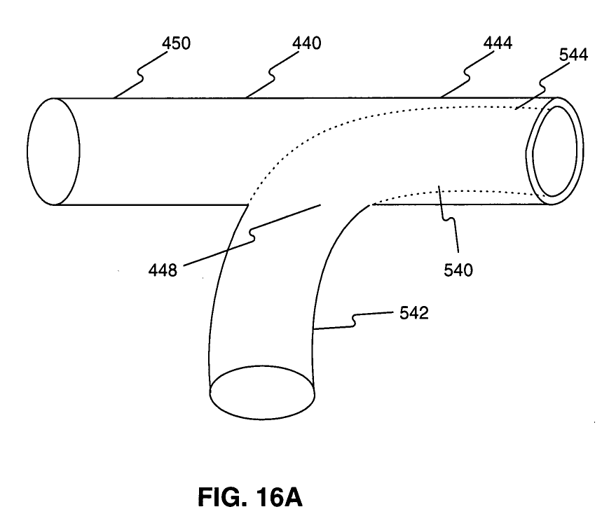

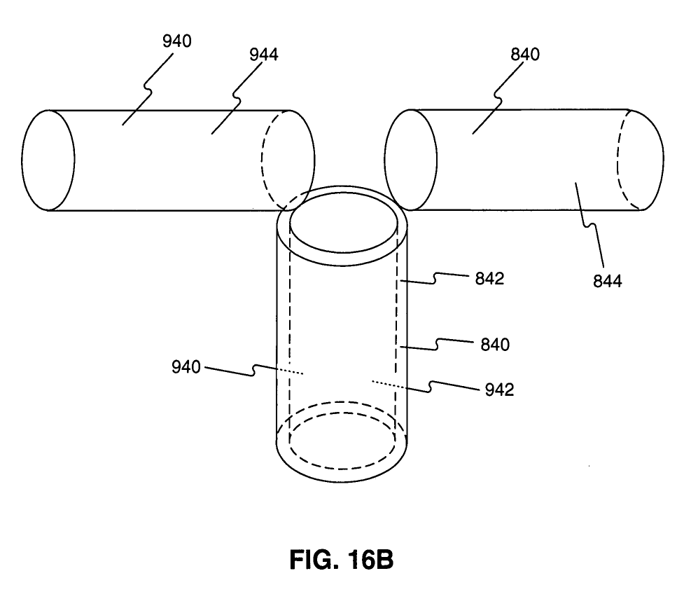

FIG. 16A shows one example of such a configuration where two stents 440, 540 are shown. One stent 440 may form both the vessel section 444 and the protection section 450. The stent 440 may define an opening 448 between the vessel section 444 and the protection section 450. Another stent 540 may include a first portion 544 which may be inserted into one of the vessel section 444 and protection section 450, and a second portion which may form the myocardial section 542. The myocardial section 542 may be placed through the opening 448 such that the first portion 544 is disposed in either the vessel section 444 (as shown in FIG. 16A) or protection section 450 (not shown) of the stent 440. Accordingly, only one of the vessel section 444 and protection section 450 may ultimately receive the first portion 544 of stent 540.

FIG. 16B shows another example of such a configuration where two stents 840, 940 are shown. One stent 840 may form both a first section 844 (e.g., one of the vessel section and the protection section) and a myocardial section 842. The first section 844 and the myocardial section 842 may be connected, for example, by a hinge or any other connector described herein. Another stent 940 may include a first section 944 (e.g., one of the vessel section and the protection section) and another myocardial section 942. The first section 944 and the myocardial section 942 may be connected, for example, by a hinge or any other connector described herein. Myocardial section 944 may be configured to be placed in myocardial section 942.

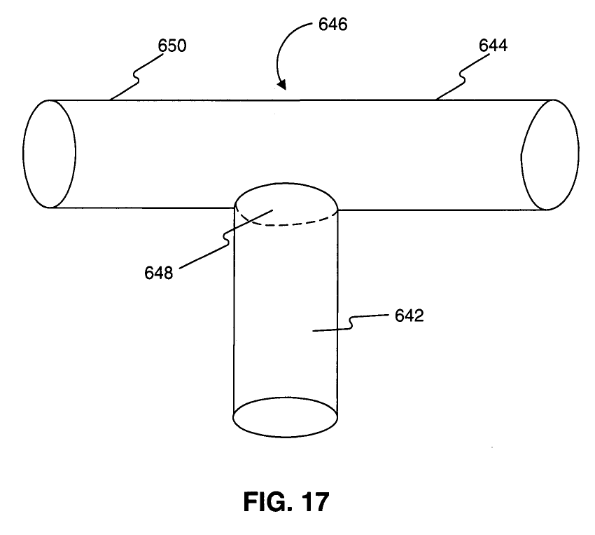

FIG. 17 shows another example of one stent 646 that forms both the combined vessel section 644 and protection section 650, like stent 440 of FIG. 16A. Stent 646 may define an opening 648, similar to opening 448 of stent 440. Myocardial section 642 may be formed separately and may be placed through opening 648. For example, myocardial section 642 may be advanced through stent 646 and out opening 648 until only a small length of section 642 adjacent to an end of myocardial section 642 is disposed in opening 648 and within stent 646. In another example, instead of section 642 being advanced through stent 646, only an end of myocardial section 642 may be placed in opening 648. Myocardial section 642 may be connected to stent 646 using any suitable method.

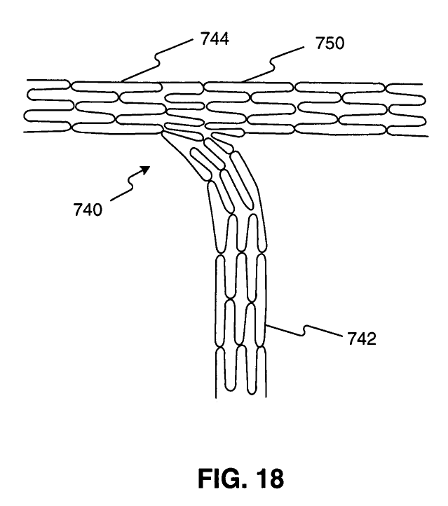

FIG. 18 shows an exemplary configuration where the vessel section 744 is integrated with the myocardial section 742 and protection section 750 into a unitary stent 740. Here, both the myocardial section 742 and the protection section 750 may be extensions of the tubing of the vessel section 744. The vessel section 744 and the protection section 750 may be fabricated first, and then the myocardial section 742 may be integrated with those sections. However, in alternative embodiments, any two of the sections may be fabricated first and then the third section may be integrated with the two sections, or all three sections may be fabricated substantially at the same time.

FIG. 23 shows an exemplary configuration of a myocardial section 1042 having a substantially cylindrical configuration connected to a vessel section 1044 having a substantially conical section 1044A and a substantially hemispherical section 1044B. The myocardial section 1042 may be connected to vessel section 1044 (e.g., a tip 1044C of substantially conical section 1044A) by a connector 1043, for example, a wire loop. The myocardial section 1042 and/or vessel section 1044 may be self-expanding and/or made of nitinol. The vessel section 1044 may be configured to anchor the myocardial section 1042 and/or occlude the coronary vessel.

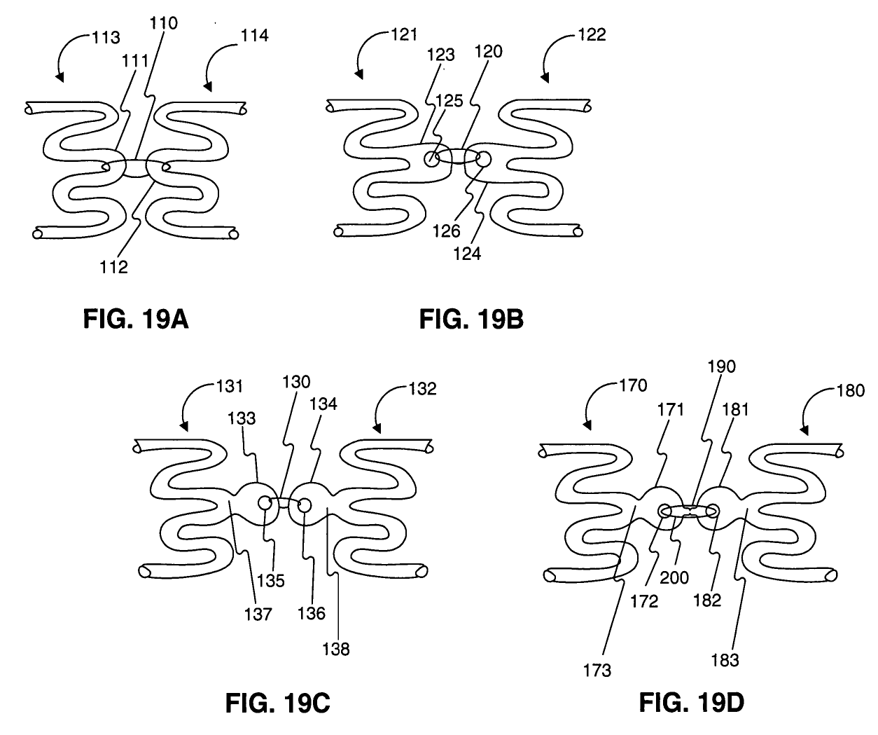

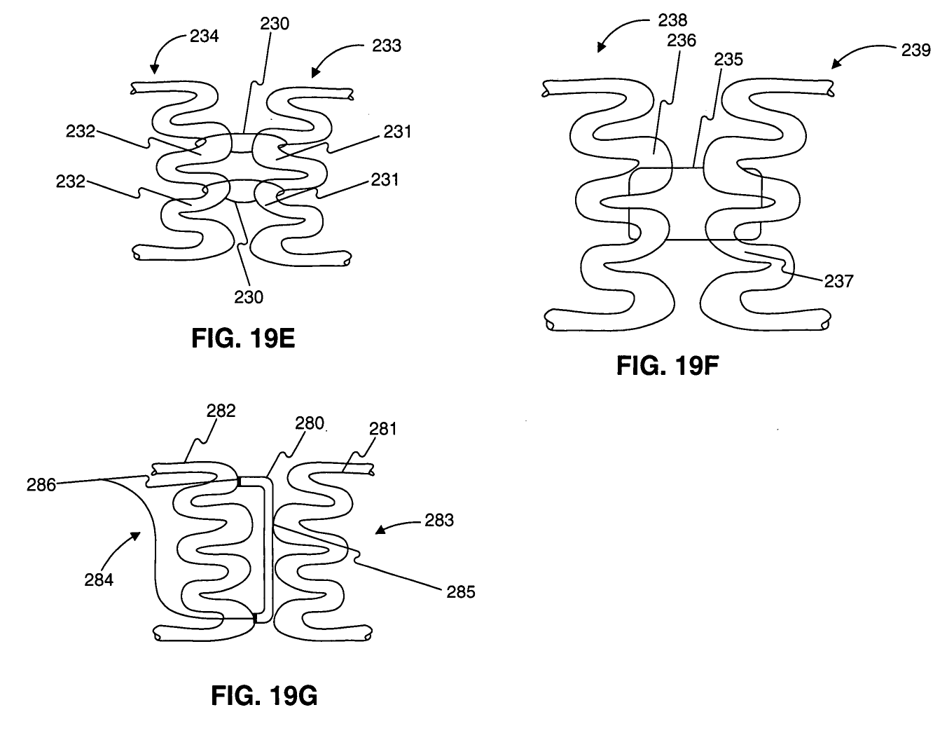

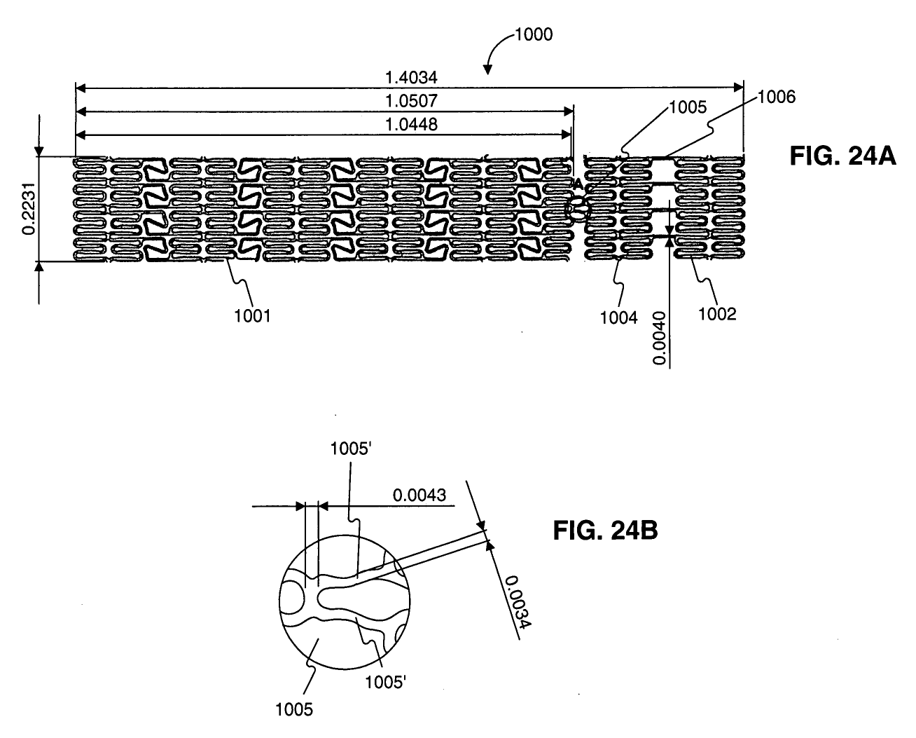

FIGS. 24A-24B show an exemplary hinged stent body 1000 in a flat and/or unrolled configuration. Hinged stent body 1000 may include a first section 1001 (e.g., a myocardial section) connected to a second section 1004 (e.g., a vessel section and/or protection section) by a connector 1005 (e.g., a notched region 190, one of connectors set forth in FIGS. 19A-19G and/or a weak hinge connector). Second section 1004 may connect to a third section 1002 (e.g., a vessel section and/or protection section) by connectors 1006. FIG. 24B depicts an enlarged view of circle A of FIG. 24A, showing connector 1005. Connector 1005 includes a pair of arms 1005′ that connect sections 1001, 1004. All dimensions in FIGS. 24A-24B are in inches. The dimensions shown are exemplary only, and other embodiments of other dimensions are within the scope of the invention.

The hinged stent body 1000 shown in FIGS. 24A-24B may be formed from a solid tube. The tube may have an outer diameter of about 1.8 millimeters and a wall thickness of about 0.20 millimeters. In another example, the wall thickness of the tube may be about 0.0063 inches. These dimensions are exemplary only, and other embodiments of other dimensions are within the scope of the invention. The tube may be a 316L stainless steel tubing, and may have an electropolished surface finish. The body 1000 should be free from contaminants such as oil, dirt, dust, chips, and residue from post-processing steps. All surface and edges of the body 1000 should be smooth and be substantially free of slag, burrs, scratches, and gouges.

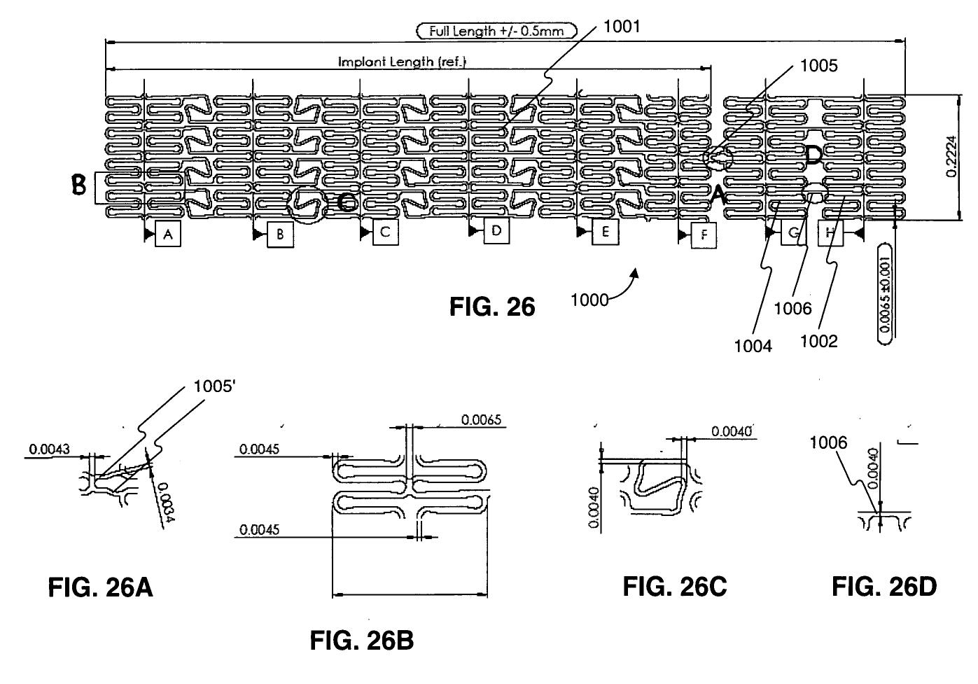

FIGS. 26 and 26A-26D show another exemplary hinged stent body 1000 in a flat and/or unrolled configuration. Hinged stent body 1000 may include a first section 1001 (e.g., a myocardial section) connected to a second section 1004 (e.g., a vessel section and/or protection section) by a connector 1005 (e.g., a notched region 190, one of connectors set forth in FIGS. 19A-19G and/or a weak hinge connector). Second section 1004 may connect to a third section 1002 (e.g., a vessel section and/or protection section) by connectors 1006. FIG. 26A depicts an enlarged view of circle A of FIG. 26, showing connector 1005. Connector 1005 includes a pair of arms 1005′ that connect sections 1001, 1004. FIG. 26B depicts an enlarged view of circle B of FIG. 26 which includes a single cell. FIG. 26C depicts an enlarged view of circle C of FIG. 26, showing a portion of the hinged stent body 1000. FIG. 26D depicts an enlarged view of circle D of FIG. 26, showing a connector 1006. All dimensions in FIGS. 26 and 26A-26D are in inches unless otherwise indicated. The dimensions shown are exemplary only, and other embodiments of other dimensions are within the scope of the invention.

Hinged stent body 1000 may have any suitable dimensions. For example, hinged stent body 1000 may have a full length, as defined in FIG. 26, between about 32.4 mm and about 37.0 mm. In another example, hinged stent body 1000 may have an implant length, as defined in FIG. 26, between about 23.4 mm and about 28.0 mm. In a further example, hinged stent body 1000 may include between about 7 cells and about 8 cells in a length direction and about 4 cells in a width direction, with an example of a single cell being shown in FIG. 26B.

FIG. 26 shows various cells labeled A-H. A width of a cell may be measured as shown by the distance set forth in FIG. 26B. Each of the cells A-H may have a suitable width. For example, one or more of cells A-H may have a width between about 0.1215 inches and about 0.152 inches (e.g., about 0.1215 inches, about 0.130 inches, about 0.138 inches, about 0.142 inches, about 0.150 inches, and about 0.152 inches).

The hinged stent body 1000 shown in FIGS. 26 and 26A-26D may be formed from a solid tube. The tube may have an outer diameter of about 1.8 millimeters and a wall thickness of about 0.20 millimeters. In another example, the wall thickness of the tube may be about 0.0063 inches. The hinged stent body 1000 may have one or more of the following properties and/or dimensions: an ultimate tensile strength of greater than or equal to about 84,000 psi; a yield strength, with a 0.2% offset, greater than or equal to about 38,000 psi; an elongation percentage, for an about 1 inch gage length, greater than or equal to about 40%; a grain size of about ASTM GS #10 or finer; a uniform, equal-axial fine grained austenitic structure; and the area including a large grain (e.g., grains with a grain size twice than that of the average grains) may be less than about 2% of any selected area. The tube may be a 316L stainless steel tubing, and may have an electropolished surface finish. The body 1000 should be free from contaminants such as oil, dirt, dust, chips, and residue from post-processing steps. All surface and edges of the body 1000 should be smooth and be substantially free of slag, burrs, scratches, and gouges. These properties and dimensions are exemplary only, and other embodiments of other properties and dimensions are within the scope of the invention.

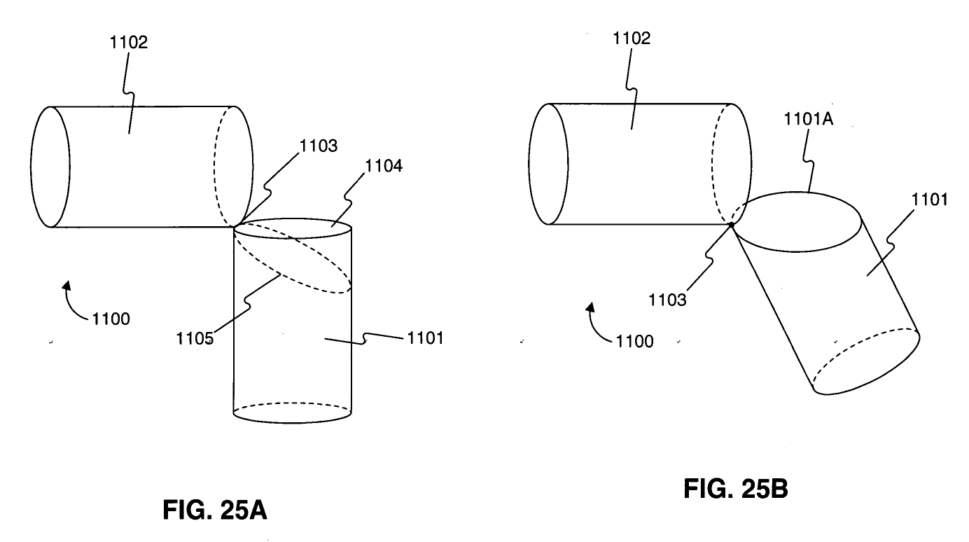

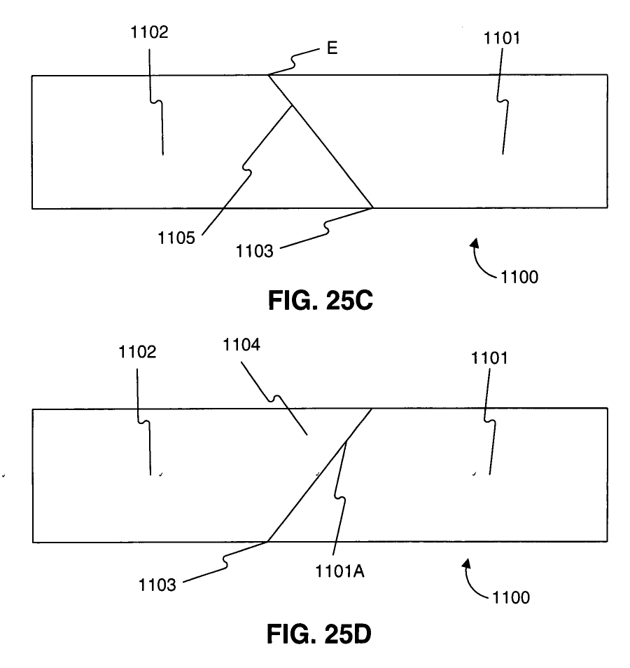

FIGS. 25A-25D show exemplary configurations of an implant 1100 including a first section 1101 (e.g., a myocardial section) and a second section 1102 (e.g., a vessel section) connected by a connector 1103. As shown in FIG. 25A, first section 1101 may include a beveled portion 1104 to be removed, for example, by cutting along dotted line 1105. FIG. 25B depicts first section 1101 without the beveled portion 1104. The geometry of the beveled portion 1104 may be determined, for example, by the angle that second section 1102 may be disposed with respect to first section 1101 when the implant is placed in the body. For example, the beveled portion 1104 may be configured such that, when removed, an edge 1101A of first section 1101 may be substantially aligned with a wall of a coronary vessel in which second section 1102 is disposed. The beveled portion 1104 may be removed so that edge 1101A may have any suitable angle with respect to a longitudinal axis of first section 1101. In some embodiments, the beveled portion 1104 may not be removed, but instead may form a part of second section 1102, for example, as shown in FIG. 25D, by cutting along a line of implant 1100 corresponding to edge 1101A.

In another example, a high distal edge E may be formed on first section 1101 by cutting implant 1100 along line 1105 at an angle so as to form second section 1102 and first section 1101 connected by connector 1103, for example, as shown in FIG. 25C. The angle of the cut may be determined, for example, by the angle that second section 1102 may be disposed with respect to first section 1101 when the implant is placed in the body. Such a high distal edge E may be desirable, for example, to prevent the heart wall from covering high distal edge E.

FIGS. 19A-19G disclose exemplary embodiments of various connectors that can be used to connect any of the various stent sections in any of the embodiments set forth herein. The connector may be one or more of a ring, loops, a wire, a strip, a string, a cable, a suture, a rope, and/or other types of connectors. Any of these connectors may be made of metal, a polymer, or any other suitable biocompatible material. Any combination of one or more of the various connectors may be used to join the various stent sections.

As shown in FIG. 19A, the connector 110 (e.g., a metal loop or a wire rope) may be placed around U-shaped portions 111, 112 (e.g., struts) of one or more various stent sections 113, 114, for example, the vessel section and the myocardial section. Such a connector 110 may behave like a ball joint. In another example, as shown in FIG. 19B, one or more stent sections 121, 122 may have end struts 123, 124 with holes 125, 126 in them, and the connector 120 may be placed through one or more of the holes 125, 126. As shown in FIG. 19C, the one or more stent sections 131, 132 may have extended grip regions 133, 134 with holes 135, 136 that are respectively connected to struts of the stent sections, 131, 132 via extensions 137, 138. The connector 130 may be placed through one or more of the holes 135, 136. As shown in FIG. 19E, two connectors 230 (e.g., a metal loop or a wire rope) may each be placed around respective U-shaped portions 231, 232 (e.g., struts) of one or more stent sections 233, 234, for example, the vessel section and the myocardial section. Such a combination of connectors 230 may behave like a hinge joint. In various embodiments, however, any number of connectors may be placed around any number of respective U-shaped portions. For example, as shown in FIG. 19F, a connector 235 may be placed through more than one U-shaped portion 236, 237 (e.g., struts) of one or more stent sections 238, 239, for example, the vessel section and the myocardial section.

As shown in FIG. 19G, connector 280 (e.g., a wire rope) may be attached (e.g., via welding or an adhesive) to U-shaped portions 281, 282 (e.g., struts) on one or more stent sections 283, 284, for example, the vessel section and the myocardial section. One or more portions of connector 280 may be attached to U-shaped portions 281, 282 at one or more locations 285, 286.

FIG. 19D shows an exemplary embodiment where one or more of the various stent sections may be connected to each other using more than one connector, for example, a temporary connection that disconnects after implantation and a connector of the types described above that remains after implantation to allow articulation between the various connected stent sections.

For example, the stent sections 170, 180 may have extended grip regions 171, 181, with holes 172, 182, respectively connected to struts of the stent sections 170, 180 via extensions 173, 183. The ends of the extended grip regions 171, 181 also may be connected to each other via a notched connection region 190. The extended grip regions 171, 181 may be connected by fabricating the stent sections 170, 180 from the same piece of metal and machining the notched region 190 in such a configuration. Alternatively, the stent sections 170, 180 may be fabricated separately and the stent sections 170, 180 may subsequently be joined together so as to form the notched region 190. Another connector 200 may be disposed through the holes 172, 182.

The notched region 190 may be used as a weak hinge connector. The notched region 190 may allow stent sections 170, 180 to articulate relative to each other without necessarily breaking. The notched region 190 may assist in the deployment of the stent sections 170, 180 in a connected (e.g., substantially straight) configuration, for example, as the stent sections 170, 180 make their way into the body through a coronary vessel, by allowing alignment of the stent sections 170, 180 relative to each other. Once the stent sections 170, 180 have been positioned in the appropriate portion of the coronary vessel and/or myocardium, the notched region 190 may allow the stent sections 170, 180 to articulate with respect to each other. Over time, continued bending of notched region 190, for example, as stent sections 170, 180 articulate relative to each other, may cause notched region 190 to fatigue and break. Even if this were to occur, however, stent sections 170, 180 may still be connected via connector 200.

There are several advantages to using connectors set forth in FIGS. 19A-19G. For example, manufacturing a stent of stent sections and a connector out of a single, integral piece of metal may be undesirable because such an integral connector may be subject to significant fatigue stresses due to the relative movement of the stent sections. Such fatigue may eventually result in a failure of the solid, integral connector after the stent is deployed in the body. As a result, the stent sections may migrate in the coronary vessel and/or heart chamber, and sharp edges of the stent of metal may injure body tissue. By using connectors set forth in FIGS. 19A-19G, such metal fatigue is significantly reduced or altogether not a factor.

In another example, at least some of the connectors set forth in FIGS. 19A-19G allow the stent sections to rotate with respect to each, providing as many as three degrees of rotational freedom. Allowing such rotation may allow for easier placement of the stent in the coronary vessel and/or myocardium by making it easier to conform the stent sections to the geometry of the body lumen. Allowing such rotation may also reduce the amount of stress between the stent and the body tissue contacting the stent when the stent is deployed.

In addition to the various delivery steps discussed above, any type of delivery approach used could include a step of measuring the heart wall thickness and determining the appropriate stent size based on the measured thickness. Such measuring could be done by numerous means apparent to those skilled in the art, including using various imaging techniques, wave generator (e.g., pressure monitoring) techniques, insertion of a device, such as a needle, for example, with graduated markings and a mechanism for observing blood flow through the device to determine it has reached the chamber and/or vessel, and other suitable measuring techniques.

Moreover, as discussed above, a vessel blocking device may be implanted in the vessel in conjunction with a hinged implant. In such cases, it may be desired to measure various vessel flow parameters, such as pressure, for example, and choose a blocking device based on the measured flow parameters. For examples of blocking devices and implanting such devices into coronary vessels, reference is made to U.S. Patent Application Publication No. 2005-0070993 A1, incorporated herein.

Alternatively, the stent may be delivered to the heart wall and coronary vessel from the heart chamber. It should also be appreciated that the myocardial section of the stent need not be positioned perpendicular to the longitudinal axis of the coronary vessel, but could be positioned at a different angle.

Further, prior to delivering the myocardial section into the heart wall MYO, an optional step may be used to provide a passageway within the heart wall MYO. The passageway, if formed, may facilitate the insertion of the myocardial section. A variety of techniques may be employed to form such a passageway including, but not limited to, using ablation techniques, dilation techniques, including balloon dilation or the insertion of a series of dilation catheters, for example, and other tissue removal techniques.

The various hinged implants described herein may be made using a variety of techniques. For example, in an exemplary aspect, when the hinged implants are in the form of stents, conventional techniques, such as, for example, laser etching and/or chemical etching of solid tubes may be used to form the stent, and/or welded wire structures may be used.

As noted above, various methods and delivery tools may be used to deliver and insert an implant into different body parts. Skilled artisans may use any embodiments, modifications and variations thereof, and other conventional techniques to deliver and insert any implant of the invention. In other words, skilled artisans may use delivery or insertion techniques known in the art to deliver or insert the devices according to embodiments of the invention.

It will be apparent to those skilled in the art that various modifications and variations can be made in the exemplary devices and methods described above and in the construction of those devices and methods. Other embodiments of the invention will be apparent to those skilled in the art from consideration of the specification and practice of the invention disclosed herein. It is intended that the specification and examples be considered as exemplary only.

1. A device for treating a heart, comprising:
a stent having a portion thereof that does not extend completely around the circumference of the stent so as to form a first stent section and a second stent section that articulate relative to each other.

2. The device of claim 1, wherein the first stent section comprises a myocardial section configured to be positioned in a heart wall between a coronary vessel and a chamber of the heart and the second stent section comprises a vessel section configured to be positioned in the coronary vessel.

3. The device of claim 2, wherein the myocardial section is configured to provide flow communication between the heart chamber and the coronary vessel when the stent is positioned in the heart wall.

4. The device of claim 2, wherein the myocardial section is configured to be positioned in the heart wall between a left ventricle and one of a coronary artery and a coronary vein.

5. The device of claim 1, further comprising a covering over at least a portion of the stent.

6. The device of claim 5, wherein the covering comprises one of a polymer, a metal, and a tissue.

7. The device of claim 1, wherein the first section and the second section are connected to each other via a hinged section.

8. The device of claim 7, wherein the portion of the stent that does not extend completely around the circumference of the stent forms the hinged section.

9. The device of claim 8, wherein the hinged section has a length equal to approximately half of a length of a stent cell.

10. The device of claim 1, wherein the portion extends from approximately 10 degrees to approximately 90 degrees around the circumference of the stent.

11. The device of claim 1, wherein the stent is expandable.

12. The device of claim 2, wherein the vessel section is configured to achieve a diameter approximately equal to a diameter of the vessel lumen when the vessel section is positioned in the coronary vessel.

13. The device of claim 1, wherein the device is configured to be percutaneously delivered and implanted in the heart.

14. The device of claim 1, wherein the device is configured to be surgically delivered and implanted in the heart.

15. A method of treating a heart, comprising:
providing a stent having a portion that does not extend completely around the circumference of the stent so as to form a first stent section and a second stent section that articulate relative to each other;
delivering the stent to a location proximate to a heart wall;
inserting the first stent section in a heart wall between a coronary vessel and a chamber of the heart; and
inserting the second stent section in the coronary vessel.

16. The method of claim 15, further comprising forming a passageway in the heart wall between the coronary vessel and the heart chamber and inserting the first section into the passageway.

17. The method of claim 15, further comprising flowing blood through the stent between the heart chamber and the coronary vessel after inserting the stent.

18. The method of claim 15, wherein delivering the stent comprises delivering the stent to the location proximate to the heart wall via a catheter.

19. The method of claim 15, wherein inserting the first stent section comprises inserting the first stent section in the heart wall between a left ventricle and one of a coronary artery and a coronary vein.

20. The method of claim 15, further comprising expanding the stent.

21. The method of claim 20, wherein expanding the stent includes expanding the stent via a balloon.

22. The method of claim 20, wherein expanding the stent includes allowing the stent to self-expand.

23. The method of claim 15, wherein delivering the stent includes percutaneously delivering the stent.

24. The method of claim 15, wherein delivering the stent includes delivering the stent through a lumen of the coronary vessel.

25-48. (canceled)

49. A device for treating a heart, comprising:
a stent comprising a first stent section, a second stent section, and a third stent section connecting the first and second stent sections,
wherein each of the first stent section and the second stent section includes at least one stent cell, and wherein the connecting section includes at least one stent segment extending around only a portion of a circumference of the stent so that the first and second stent sections articulate relative to each other.

50. The device of claim 49, wherein a stent cell includes one of a plurality of repeating annular segments of the stent.

51. The device of claim 49, wherein the connecting section has a length less than a length of a stent cell.

52. A device for treating a heart, comprising:
a stent structure having a circumferential portion missing therefrom so as to form a first section of the stent structure and a second section of the stent structure that are configured to articulate relative to each other.

53. A device for treating a heart, comprising:
a first stent section;
a second stent section connected to the first stent section and configured to articulate relative to the first stent section; and
a third stent section connected to the first stent section, unconnected to the second stent section, and configured to articulate relative to the first stent section,
wherein the first, second, and third stent sections are configured to form a substantially T-shaped structure.

54. The device of claim 53, wherein the first stent section is connected to the second stent section via a connector and the first and second stent sections are configured to articulate about the connector.

55. The device of claim 53, wherein the third stent section is connected to the first stent section via a connector and the first and third stent sections are configured to articulate about the connector.

56. The device of claim 53, wherein the first stent section is configured to be positioned in one of a coronary vessel and a heart wall between a coronary vessel and a heart chamber.

57. The device of claim 53, wherein the first stent section is configured to be positioned in a heart wall between a coronary vessel and a heart chamber, and the second and third stent sections are configured to be positioned in the coronary vessel.

58. The device of claim 53, wherein the first and second stent sections are configured to be positioned in a coronary vessel and the third section is configured to be positioned in a heart wall between the coronary vessel and a heart chamber.

59. A device for treating a heart, comprising:
a first stent section having a lumen, an open first end, an open second end, and a hole between the first and second ends; and
a second stent section extending through the hole, the second stent section having a first portion extending within the lumen of the first stent section and a second portion extending from the hole outside of the lumen.

60. The device of claim 59, wherein the first and second stent sections form a substantially T-shaped structure.

61. A device for treating a heart, comprising:
a first stent section and a second stent section connected to, and configured to articulate relative to, each other; and
a third stent section and a fourth stent section connected to, and configured to articulate relative to, each other,
wherein one of the third stent section and the fourth stent section is configured to be disposed within a lumen of one of the first stent section and the second stent section.

62. The device of claim 61, wherein a longitudinal axis of the one of the third stent section and the fourth stent section is configured to be substantially parallel to a longitudinal axis of the one of the first stent section and the second stent section.

63. A device for treating a heart, comprising:
a first stent section; and
a second stent section connected to the first stent section via a connector and configured to articulate relative to the first stent section about the connector.

64. The device of claim 63, wherein the connector is at least one of a ring, a loop, a wire, a strip, a string, a cable, a suture, and a rope.

65. The device of claim 63, wherein each of the first stent section and the second stent section includes a U-shaped section and the connector is disposed about the U-shaped section of each of the first stent section and the second stent section.

66. The device of claim 63, wherein each of the first stent section and the second stent section includes a hole and the connector is disposed through the hole of each of the first stent section and the second stent section.

67. The device of claim 63, wherein the connector is a notched region.

68. The device of claim 63, wherein the connector is attached to at least one of the first stent section and the second stent section.

69. The device of claim 63, wherein the connector is at least two connectors.

70-77. (canceled)
